(19) United States
(12) Patent Application Publication
(10) Pub. No.: US 2006/0222633 A1
(43) Pub. Date: Oct. 5, 2006

(54) Prevention, decrease, and/or treatment of immunoreactivity by depleting and/or inactivating antigen presenting cells in the host

(75) Inventors: Warren D. Shlomchik, Westport, CT (US); Mark Jay Shlomchik, Woodbridge, CT (US)

Correspondence Address: COOLEY GODWARD LLP, THE BROWN BUILDING - 875 15TH STREET, NW, SUITE 800, WASHINGTON, DC, 20005-2221, US

(73) Assignee: Yale University

(21) Appl. No.: 11/377,647

(22) Filed: Mar. 17, 2006

Related U.S. Application Data
(63) Continuation-in-part of application No. 09/568,834, filed on May 11, 2000.
(60) Provisional application No. 60/663,371, filed on Mar. 17, 2005.

(51) Int. Cl. A61K 35/14; A61K 48/00; A61K 39/02; A61K 36/47

(52) U.S. Cl. 424/93.7; 424/133.1; 424/235.1; 424/731; 424/93.21

(57) ABSTRACT

The invention includes compositions and methods for depleting and/or inactivating antigen presenting cells, or for otherwise impairing the biological function of antigen presenting cells, which compositions are useful for treatment of graft versus host disease and other immune diseases.

### CROSS REFERENCE TO RELATED APPLICATIONS

This application is a continuation-in-part of U.S. application Ser. No. 09/568,834, filed May 11, 2000, the contents of which are incorporated herein by reference in its entirety for all purposes. This application also claims priority pursuant to 35 U.S.C. §119(e) to U.S. provisional patent application No. 60/663,371, which was filed on Mar. 17, 2005, the contents of which are incorporated herein by reference in its entirety for all purposes.

### STATEMENT REGARDING FEDERALLY SUPPORTED RESEARCH OR DEVELOPMENT

This invention was supported in part by funds obtained from the U.S. Government (National Institutes of Health Grant Numbers CA-096943, R01 HL66279) and the U.S. Government may therefore have certain rights in the invention.

### FIELD OF THE INVENTION

The field of the invention is depletion and/or inactivation of antigen presenting cells. The invention relates to methods of preventing, ameliorating, decreasing, and/or treating graft versus host diseases (GVHD) in a host mammal by means of depleting, inhibiting and/or inactivating donor and/or host antigen presenting cells (APCs).

### BACKGROUND OF THE INVENTION

Graft-versus-host disease (GVHD) is an increasingly common complication of allogeneic stem cell transplantation (alloSCT). In particular, GVHD occurring after the establishment of donor hematopoietic chimerism is on the rise, affecting up to 80% of patients in some series. The rise in incidence is likely multifactorial and may be influenced by the greater number of patients at risk because of better supportive care, use of peripheral blood stem cells, delayed leukocyte infusion, nonmyeloablative alloSCT, and early withdrawal of immunosuppression (Lee et al. 2002; Anderson et al. 2003; Atkinson et al. 1990; Castagna et al. 2002; Flowers et al. 2002; Remberger et al. 2002; Zecca et al. 2002). Most strategies for preventing and treating GVHD are directed toward depleting or impairing the function of donor T cells. Yet despite the introduction of new anti-GVHD therapies, including those that target tumor necrosis factor-alpha (TNF-α) and interleukin-2 receptor (IL-2R)-expressing cells, GVHD continues to be a serious problem. Therefore, a better understanding of the induction and pathogenic mechanisms of GVHD, both acute and chronic, is needed.

GVHD is initiated when alloreactive T cells are primed by professional antigen-presenting cells (APCs) to undergo clonal expansion and maturation. Studies have focused on the mechanics of antigen presentation, which are a crucial initial step and a potential therapeutic target. For a period of time following alloSCT, patients are chimeric for host and donor APCs (Auffermann-Gretzinger et al. 2002; Clark et al. 2003; Imamura et al. 2003; Nachbaur et al. 2003). Previously, the inventors showed that radiation-resistant host APCs are required for CD8-mediated GVHD across minor histocompatibility antigens (miHAs) and for GVHD mediated by both CD4 and CD8 cells across major histocompatibility complex (MHC) mismatches (Shlomchik et al. 1999; Ruggeri et al. 2002; U.S. patent application Ser. No. 09/568,834, the application from which the instant application claims priority).

There is a long felt need in the art for the development of specific and improved mechanisms for reducing GVHD in animals, particularly in humans. The present invention satisfies this need.

### SUMMARY OF THE INVENTION

Prior studies left open whether recipient APCs are also important for CD4-mediated GVHD across only minor H antigens. Observations that the MHCII antigen processing pathway can efficiently present exogenously acquired antigens led us to consider whether donor-derived APCs may be important. Nonetheless, the inventors postulated that there are reasons to believe that recipient APCs may be particularly important for CD4-mediated GVHD across only minor Ha antigens: 1) endogenous antigens can be presented on MHCII and donor-derived APCs would not have access to this antigen pool (Brooks et al. 1993; Bogen et al. 1993; Bodmer et al. 1994; Lechler et al. 1996); 2) long-term resident recipient APCs present at the time of transplant may present more exogenously acquired host antigen than engrafting donor APCs; and 3) T cell activation may be more efficient early post transplant and substantial numbers of donor-derived APCs may not yet be present. In the present invention we demonstrate that recipient APCs are indeed important for CD4-mediated GVHD across only minor H antigens.

Although host APCs are critical in the aforementioned models, we hypothesized that donor APCs may be also be important. Because GVHD can occur after the achievement of donor hematopoietic engraftment, it may be initiated, or at least progress, when hematopoiesis from donor-derived cells predominates (Clark et al. 2003). This suggested to us an important if not obligate role for donor-derived APCs. These donor-derived APCs would prime donor T cells by acquiring exogenous host antigens and presenting them on MHCI and MHCII. The MHCII antigen processing pathway is efficient in presenting exogenously acquired antigens and thus one might predict an important role for donor-derived APCs (Germain et al. 1994; Lanzavecchia et al. 1996; Sant et al. 1994; Wolf et al., 1995). However, exogenously acquired antigens can also presented on MHCI, though in general this process is less efficient. Thus, while host APCs are required for CD8-mediated GVHD across only minor H antigens, donor-derived APCs may nonetheless be important.

Experiments to date have not yet determined which source(s) of APCs, donor or host, are primarily responsible for GVHD after it is initiated. The resolution of this issue is of great practical as well as theoretical importance. Understanding the roles of donor and host APCs in GVHD induction will direct distinct and novel strategies for reducing acute and chronic GVHD, by specifically targeting either or both populations.

The present invention relates to methods for preventing, ameliorating, decreasing, and/or treating graft versus host diseases (GVHD) by targeting antigen presenting cells.

The methods of the present invention provide several advantages over previously described methods, one being that the prevention, amelioration, decrease, and/or treatment of GVHD are carried out by depleting or inhibiting both donor and host antigen presenting cells. As used herein, the words “inhibiting” and “inactivating” are used interchangeably in the application, but they are often referenced together so that this is clear to the reader that they are interchangeable.

In previously described methods, prevention and treatment of GVHD are achieved by depleting host antigen presenting cells with an antigen presenting cell depleting composition to effect impairment of antigen presenting cell function or killing of the antigen presenting cells. While such methods have been shown to be effective in reducing acute GVHD occurrence, it is less clear that the methods are equally effective in preventing, ameliorating, decreasing or treating established GVHD. The present invention is based on the discovery that GVHD is not only dependent on recipient APCs but that donor APCs have a nonredundant role in contributing to GVHD. The implication of this finding is that depletion or inactivation of donor-derived APCs is effective in treating established GVHD. The inventors have shown that donor APCs contribute to CD8-mediated GVHD across only minor H antigens and yet donor APCs are not required for CD8-mediated graft-versus-leukemia (GVL) (Matte et al. 2004; additional unpublished data). The result that donor APC impairment did not affect GVL suggested to us that eliminating donor APCs to treat ongoing GVHD may not compromise GVL against chronic phase chronic myelogenous leukemia (CP-CML). The present invention is further based on the discovery that impairment of either donor or host APCs diminished CD4-mediated GVHD across only minor H antigens. These results suggest that strategies that target either donor- or host-derived APCs may mitigate the manifestations of CD4 and CD8-dependent GVHD and in sum these data provide a strong rationale for targeting both donor and host APCs, rather than just host APCs.

The present invention also provides methods of preventing GVHD by depleting and/or inhibiting/inactivating antigen presenting cells in donor cells prior to or after their transplantation into a host. The depletion and/or inhibition/inactivation of APCs in the donor cells prior to transplantation can be accomplished in vitro and/or in vivo.

The present invention further provides methods of treating GVHD by depleting and/or inhibiting/inactivating antigen presenting cells at any time after transplantation during which donor and/or host antigen presenting cells are present. In the instance of treating GVHD using the methods of the present invention, one could administer an antigen presenting cell depleting composition at any time during which or after which GVHD has begun or is suspected of having begun.

The present invention also provides methods whereby one can target the donor-derived dendritic cells by administering the depleting and/or inhibiting/inactivating composition at any time post transplantation of the donor cells into the host but before GVHD has begun. Such methods would be considered prevention methods according to the present invention.

The present invention provides methods of preventing, ameliorating, decreasing, and/or treating GVHD in a host mammal by depleting antigen presenting cells in a population of hematopoietic cells of the host with an antigen presenting cell depleting and/or inhibiting/inactivating composition following transplantation of the donor cells, wherein said GVHD is prevented, ameliorated, decreased, and/or treated in said host mammal by virtue of said depletion of said antigen presenting cells.

In one aspect, the method comprises the steps of (a) transplanting hematopoietic cells from a donor mammal to a host mammal; and (b) depleting antigen presenting cells in a population of hematopoietic cells in said host mammal with an antigen presenting cell depleting and/or inhibiting/inactivating composition. In methods of prevention, the composition can be administered prior to onset of GVHD; while in methods of treating, the composition can be administered following onset of GVHD.

In some instances it may be desirable and/or necessary to utilize both the prevention and treatment methods of the present invention for a host that receives the donor cells.

In one aspect, the antigen presenting cells are selected from the group consisting of dendritic cells, B lymphocytes and macrophages, monocytes, CD34⁺ cells, fibroblasts, stem cells, and cheratinocytes.

In one aspect, the depleting is performed in vivo in a mammal.

In one aspect, the hematopoietic cells are human hematopoietic cells.

In one aspect, the antigen depleting or inhibiting/inactivating composition is selected from the group consisting of a toxin, an antibody, a radioactive molecule, a nucleic acid, a peptide, a peptidomemetic and/or a ribozyme.

In one aspect, the toxin is an immunotoxin. Examples of such toxins include but are not limited to saporin, ricin, diptheria toxin and pseudomonas exotoxin A.

In one aspect, the antibody is selected from the group consisting of antibody specific for CD1a, antibody specific for CD11c, antibody specific for MHCII, antibody specific for CD11b, antibody specific for DEC205, antibody specific for B71, antibody specific for B72, antibody specific for CD40, antibody specific for Type I lectins and antibody specific for Type II lectins.

In one aspect, the nucleic acid molecule is selected from the group consisting of a gene and an oligonucleotide.

In one aspect, the radioactive molecule is a radioactively labeled antibody.

In another aspect, the antigen depleting and/or inhibiting/inactivating composition is a chimeric composition comprising an antibody and a toxin (e.g., saporin conjugated to an antibody).

In yet another aspect of the invention, the antigen depleting and/or inhibiting/inactivating composition is delivered to the antigen presenting cell in a vector such as a viral vector and a non-viral vector.

Further included in the invention is a method of preventing graft versus host disease in a mammal. The method comprises obtaining a population of hematopoietic stem cells from the mammal, adding to the cells a gene which when expressed in the cells is capable of killing the cells, selecting cells having the gene incorporated therein, irradiating the mammal to remove bone marrow cells in the mammal, adding the selected cells to the mammal, inducing expression of the gene in the selected cells in the mammal thereby effecting killing of antigen presenting cells in the mammal, providing the mammal with an allogeneic bone marrow transplant, wherein graft versus host disease is prevented, ameliorated, decreased and/or treated in the mammal by virtue of the killing of the antigen presenting cells.

In one embodiment, the gene is operably linked to an inducible promoter and expression of the gene is effected by administration of an inducer of the promoter to the mammal. In another embodiment, the gene encodes a toxin.

Also included is a method of preventing graft versus host disease in a mammal which includes obtaining a population of hematopoietic stem cells from the mammal, adding to the cells a gene which when expressed in the cells in the presence of a corresponding agent is capable of killing the cells, selecting cells having the gene incorporated therein, irradiating the mammal to remove bone marrow cells in the mammal, adding the selected cells to the mammal, adding the corresponding agent to the mammal to effect killing of the selected cells in the mammal thereby effecting killing of antigen presenting cells in the mammal, providing the mammal with an allogeneic bone marrow transplant, wherein graft versus host disease is prevented, ameliorated, decreased and/or treated in the mammal by virtue of the killing of the antigen presenting cells.

In one embodiment, the gene is thymidine kinase, the gene is operably linked to a constitutive promoter and the corresponding agent is ganciclovir.

In another embodiment, the gene is thymidine kinase, the gene is operably linked to an inducible promoter, the corresponding agent is ganciclovir, and prior to adding the corresponding agent to the mammal, the expression of the gene is induced by administration of an inducer of the promoter to the mammal.

Additional aspects and embodiments of the present invention will be obvious to one skilled in the art upon reviewing the present description, wherein such obvious variants are within the scope of the invention.

### BRIEF DESCRIPTION OF THE DRAWINGS

FIG. 1 is a diagram depicting the experimental protocol for the experiments presented in Example 1 herein. Eight to ten week C57BL/6 (H-2^{b}) mice received 200 μg of NK1.1 moAB intraperitoneally on days-2 and -1 to deplete natural killer cells and facilitate engraftment of B2M^{-/-} cells (Bix et al., 1991, Nature 349:329331). On day 0, mice received two 500 cGy fractions separated by 3 hours from a dual cesium radiator, followed by injection with 10⁷ T cell depleted bone marrow from C57BL/6 beta-2-microglobulin knock-out mice (β₂M^{-/-} T⁻BM) or wild type C57BL/6 (β₂M^{+/+} T⁻BM). Mice received acidified water and were kept in microisolator cages. Four months after the first transplant, chimeras were re-irradiated twice with 375 cGy fractions separated by 3 hours. Mice were then injected with 5-7×10⁶ T cell depleted bone marrow cells obtained from C3H.SW (H-2^{b}) mice (C3H.SW T⁻BM) with or without 1× or 2×10⁶ purified C3H.SW CD8⁺ T cells. Mice were examined for the development of GVHD.

FIG. 2 is a series of images depicting the fact that B6→B6 CD8 recipients develop GVHD. FIG. 2A depicts clinical GVHD. Representative β₂M^{-/-}→B6 C3H.SW T⁻ BM and CD8 recipient (left) and B6→B6 C3H.SW T⁻BM and CD8 recipient (right) from experiment 1 are shown. FIG. 2B (comprising Panels A-H) depicts the histology of the mice. Representative β₂M^{-/-}→B6 (Panels B, D and F) and B6→B6 (Panels A, C, E, G and H) recipients of C3H.SW BM and CD8⁺ T cells are shown. Liver (Panels A and B); small intestine (Panels C and D); skin (Panels E and F). Note periportal mononuclear infiltrates in Panel A; apoptotic cells in small bowel crypts in Panel C (arrow); and mononuclear cell infiltrate, fibrosis, epidermal maturation disarray, and necrotic keratinocytes (arrow) in Panel E. These changes were absent in β₂M^{-/-}→B6 recipients. Panel G, horseradish peroxidase staining of CD8 positive cells; note CD8 cells invading follicles and epidermis (arrows). Panel H: immunohistochemical staining for CD4 cells from the same mouse as in Panel G. Note the paucity of cells staining for CD4 relative to CD8 in Panel G.

FIG. 3A is a graph depicting the percent weight loss in mice. Mice were individually weighed three times per week, beginning on day 0. Mean weights of mice in Experiment 1 were plotted as percent weight change versus time. The groups are indicated on the figure. When CD8 cells were included as indicated on the Figure, 1×10⁶ cells were used.

FIG. 3B is a graph depicting the survival of the mice. In a second experiment, recipients of a second transplant were followed for survival. β₂M^{-/-}→B6 and B6→B6 chimeras were irradiated followed by the infusion of C3H.SW T⁻ BM with 0 (-▪-B6→B6 T⁻BM alone (four mice); —c—β₂M^{-/-}→B6 T⁻ BM alone (eight mice)), 1×10⁶ (-●-B6→B6 T⁻BM⁺ 1×10⁶ CD8 (eight mice); -⊖-β₂M^{-/-}→B6 T⁻BM+1×10⁶ CD8 (twelve mice)), or 2×10⁶ (-▴-B6→B6 T-BM⁺ 2×10⁶ CD8 (eight mice); Δ-β₂M^{-/-}β6 T-BM⁺ 2×10⁶ CD8 (twelve mice)) purified C3H.SW CD8⁺ T cells.

FIG. 4 are two illustrations depicting MHC I expression on macrophages, dendritic cells and B lymphocytes. FIG. 4A depicts MHC I expression of dendritic cells (DC). Dendritic cells were isolated by first collagenase treating spleens and lymph nodes followed by centrifugation through 30% BSA. Dendritic cells were identified by 4 color flow cytometry. Cells staining with a multi lineage cocktail of antibodies against Thy1.2 (T cells), Gr-1 (granulocytes), TERR 119 (erythroid), and CD45R (B220; B cells) were first excluded. Then dendritic cells that were either CD11c⁺/CD11b⁻ or CD11c⁺/CD11b⁺ were gated on separately, and MHC I expression was examined. FIG. 4B depicts MHC I expression on dendritic cell (DC), macrophage (macroφ), and B cells in lymph nodes and spleens of β₂M^{-/-}→B6 chimeras. æ individual mouse; median. 12 and 11 mice were analyzed for splenic dendritic cell chimerism; 7 mice were analyzed for macroφ and B cell chimerism.

FIG. 5 is a Table depicting histologic scoring of GVHD. Formalin fixed, paraffin imbedded sections were stained with hematoxylin and eosin, randomized and read blindly. Findings were scored according to established criteria and were given an overall interpretation of positive (+), indefinite (+/−), or negative (−) for GVHD. N=number of mice analyzed.

FIG. 6 is a series of illustrations depicting the fact that in vivo α-CD11c treatment completely stains dendritic cells. Spleens obtained from mice treated with two intraperitoneal (i.p.) injections of phosphate buffered saline (PBS; panels A-D) or 500 μg of a hamster monoclonal antibody against CD11c (clone 33D1; panels E and F) were dispersed and digested with collagenase. The light fraction enriched for dendritic cells was separated by centrifugation on 30% BSA. Dendritic cells were identified by flow cytometry. Cells staining with a multi lineage cocktail of phycoerythrin conjugated antibodies against CD3 (T cells), TERR 119 (erythroid cells), Gr-1 (granulocytes), and CD45R (B220; B cells) were first excluded. Then the remaining cells were assessed for expression of CD11b and CD11c (CD11b cy5 and biotin-CD11c with a streptavidin PerCP (SA-PerCP) second step reagent). In the PBS treated mice, CD11c⁺ dendritic cells were easily identified and expressed high levels of MHC II as expected (histograms to the right of dot plots). If the cells obtained from PBS treated mice were first incubated with purified 33D1, CD11c⁺ dendritic cells could no longer be identified using anti-CD11c (C). However, if these cells were detected using a biotin conjugated mouse α-hamster antibody followed by SA-PerCP instead of α-CD11c, they cells could again be visualized (D). Without 33D1 preincubation, staining with mouse anti-hamster antibody was negative (B). In mice treated with i.p. 33D1, direct staining with α-CD11c did not identify dendritic cells (E). However, staining with mouse α-hamster identified a population similar to that seen in A and D, suggesting that nearly all CD11c molecules on splenic dendritic cells were bound to 33D1 antibody and thus were unavailable for staining with labeled α-CD11c antibodies. Similar results were obtained using lymph node cells.

FIG. 7 shows that recipients of B2m^{-/-} bone marrow (BM) develop less GVHD. Weight change (left panels), skin GVHD incidence (middle panels) and numbers of skin ulcers (right panels) in two independent experiments (a,b). Weight change: †P<0.02, B2m^{-/-} bone marrow alone versus B2m^{-/-} bone marrow and CD8, except day 61 (P<0.03); *P<0.006, B2m^{-/-} bone marrow and CD8 cells versus wild-type bone marrow and CD8 cells, except day 33 (P<0.01); **P<0.05, B2m^{-/-} bone marrow and CD8 cells versus wild-type bone marrow and CD8 cells. (c) Pathology scores. Circles represent individual mice; lines represent means. Six recipients of C3H.SW bone marrow and CD8 cells died from GVHD and were unavailable for pathologic analysis. Wild-type bone marrow alone versus wild-type bone marrow and CD8 cells: **P<0.02; **P<0.005. B2m^{-/-} bone marrow alone versus B2m^{-/-} bone marrow and CD8 cells: †P<0.73; ††P<0.03. B2m^{-/-} bone marrow and CD8 cells versus wild-type bone marrow and CD8 cells: P<0.003; P<0.2.

FIG. 8 shows that Splenic DCs in mice with GVHD were donor-derived. DCs were identified by gating on Lin-PI-cells (a) that were CD11c⁺ (b). Residual host DCs are Ly5.1⁺ MHC I⁺; wild-type donor APCs are Ly5.1⁻ MHC I⁺; B2m^{-/-} donor APCs are Ly5.1⁻ MHC I⁻. In recipients of wild-type C3H.SW bone marrow, most DCs were donor-derived. In recipients of only B2m^{-/-} bone marrow, most DCs were donor-derived, but 1.9% were residual host cells. However, in recipients of B2m^{-/-} bone marrow and CD8 cells, nearly all or all DCs were donor-derived. BM, bone marrow.

FIG. 9 shows that MHC II⁺ cells in skin and bowel are donor-derived. Tissues were stained with anti-MHC II (red), anti-Ly5.1 (host-specific; green, a, c, h, i, k and o), anti-Ly5.2 (donor-specific; green, d, f, g, l and n), isotype for Ly5.1 (green, b and j) and isotype for Ly5.2 (green, e and m). Note the absence of recipient MHC II⁺ cells in skin (a; magnified in inset) and bowel (i) from recipients of B2m^{-/-} bone marrow and CD8 cells, as staining was similar to that seen with an isotype for anti-Ly5.1 (b and j). The failure to detect recipient Ly5.1 cells was not technical, as skin and bowel from Ly5.1 ⁺ control mice stained with anti-Ly5.1 (c, skin; k, bowel). In contrast, there was strong staining for donor derived Ly5.2⁺ MHC II⁺ cells in skin (d; magnified in inset) and bowel (I) of recipients of B2m^{-/-} bone marrow and CD8 cells. MHC II⁺ cells in skin from recipients of wild-type bone marrow and CD8 cells were Ly5.2⁺ and donor-derived (g). Ly5.2 staining was specific, as it was similar to that seen with an isotype control for anti-Ly5.2 (e, skin; m, bowel). Also, anti-Ly5.2 did not stain skin (f) and bowel (n) from Ly5.1⁺ control mice. MHC II staining was specific, as there was no staining observed in skin (h) and bowel (o) from H2-Abl^{-/-} Ly5.1⁺ MHC II⁻ mice.

FIG. 10 shows that donor APCs are not required for CD8-mediated GVL. Irradiated B6 mice were reconstituted with mouse CP-CML cells, T cell-depleted wild-type (WT) or B2m^{-/-} C3H.SW bone marrow (BM), with or without purified wild-type C3H.SW CD8⁺ T cells. Number of mice per group is in parentheses. Survival (left panel), incidence of skin disease (middle panel) and percent weight change (right panel). Incidence of skin disease, P<0.0007 comparing wild-type bone marrow and CD8 cells with B2m^{-/-} bone marrow and CD8 cells. Weights, *P<0.045 comparing B2m^{-/-} bone marrow and CD8 cells and wild-type bone marrow and CD8 cells.

FIG. 11 shows that CD80/86 expression is required for skin cGVHD. Combined data from 3 experiments are shown. On day 0 recipient mice were lethally irradiated and reconstituted with 8×10⁶ BM cells WT BM or CD80/86^{-/-} [CD80/86] BM) alone or with 2×10⁶ WT CD4 cells (BM+CD4). All BM controls (ctrls): WT or CD80/86^{-/-} recipients of WT or CD80/86^{-/-} BM (n=20); WT recipients of WT BM⁺ CD4 (n=17); WT recipients of CD80/86^{-/-} BM+CD4 (n=8); CD80/86^{-/-} recipients of WT BM+CD4 (n=28); CD80/86^{-/-} recipients of CD80/86^{-/-} BM+CD4 (n=16). (A) Incidence of cGVHD. P<0.01 for CD80/86^{-/-} recipients of WT BM+CD4 as compared with all other experimental groups. (B) Average clinical disease score for mice affected with cGVHD (unaffected mice are excluded). P<0.01 for CD80/86^{-/-} recipients of CD80/86^{-/-} BM+CD4 as compared with all other CD4 recipients. BM control mice and CD80/86^{-/-} recipients of CD80/86^{-/-} BM+CD4 did not get cGVHD and are represented on the graph as scoring “0.” (C) Pathology scores for representative mice. Mean score is indicated by a horizontal bar. P<0.01 for CD80/86^{-/-} recipients of CD80/86^{-/-} BM+CD4 as compared with all other CD4 recipients. P=0.1715 for CD80/86^{-/-} recipients of CD80/86^{-/-} BM+CD4 as compared with BM control recipients.

FIG. 12 shows that donor-type APCs are sufficient for induction of cGVHD. Combined data from 2 experiments are shown. On day 0, chimeric recipient mice (previously prepared) were lethally irradiated and reconstituted with 8×10⁶ WT BM cells alone (broken line; n=21); or WT BM plus 10⁷ WT spleen cells (host→host) (thin solid line; n=32); (donor host) (bold solid line; n=36). Data for all BM control recipients were combined. (A) Incidence of cGVHD. P<0.01 for donor host recipients versus host host recipients of spleen cells. (B) Average clinical disease score for mice affected with cGVHD (unaffected mice are excluded). BM control mice did not get cGVHD and are represented on the graph as scoring “0.” (C) Pathology scores for representative mice. Mean score is indicated by a horizontal bar.

FIG. 13 a graph elucidating differential roles of CD40 and CD80/86 on donor and host APCs in cGVHD. (A) Incidence of cGVHD in CD40^{-/-} (CD40) recipients. One representative experiment is shown. On day 0, recipient mice were lethally irradiated and reconstituted with 8×10⁶ WT BM cells alone; both recipient types (n=9); or WT BM plus 10⁷ WT spleen cells as a source of CD4 cells, WT recipients (n=15), CD40 recipients (n=14). (B) Clinical disease in CD40 recipients. Average clinical score for mice affected with cGVHD (unaffected mice are excluded). BM control mice did not get cGVHD and are represented on the graph as scoring “0.” (C) Incidence of cGVHD in CD80/86^{-/-} recipients. Combined data from 2 experiments are shown. On day 0, recipient mice were lethally irradiated and reconstituted with 8×10⁶ WT BM cells alone; both recipient types (n=19); or WT BM plus 10⁷ WT spleen cells as a source of CD4 cells, WT recipients (n=27), CD80/86^{-/-} recipients (n=29). P<0.01 for CD80/86^{-/-} recipients versus WT recipients of spleen cells. (D) Clinical disease in CD80/86^{-/-} recipients. Average clinical score for mice affected with cGVHD (unaffected mice are excluded). BM control mice did not get cGVHD and are represented on the graph as scoring “0.”*P<0.05 for CD80/86^{-/-} recipients as compared with WT recipients of spleen cells. (E) Incidence of cGVHD in CD80/86^{-/-} recipients of CD40^{-/-} BM. Combined data from 2 experiments are shown. On day 0, recipient mice were lethally irradiated and reconstituted with 8×10⁶ WT or CD40^{-/-} BM cells alone; both BM types (n=22), WT BM plus 2×10⁶ purified CD4 cells, WT recipients (n=15), CD80/86^{-/-} recipients (n=32); or CD40^{-/-} BM plus 2×10⁶ purified CD4 cells, CD80/86^{-/-} recipients (n=34). P<0.01 for CD80/86^{-/-} recipients of CD40^{-/-} BM+CD4 cells versus WT BM+CD4 cells. (F) Clinical disease in CD80/86^{-/-} recipients of CD40^{-/-} BM. Average clinical score for mice affected with cGVHD (unaffected mice are excluded). BM control mice did not get cGVHD and are represented on the graph as scoring “0.” (G) Incidence of cGVHD in WT recipients of CD40^{-/-} BM. One representative experiment of 2 is shown. On day 0, recipient mice were lethally irradiated and reconstituted with 8×10⁶ WT or CD40^{-/-} BM cells alone; both BM types (n=10), WT BM plus 2×10⁶ purified CD4 cells (n=15), or CD40^{-/-} BM plus 2×10⁶ purified CD4 cells (n=15). P<0.01 for recipients of CD40^{-/-} BM+CD4 cells versus WT BM+CD4 cells. (H) Clinical disease in WT recipients of CD40^{-/-} BM. Average clinical score for mice affected with cGVHD (unaffected mice are excluded). BM control mice did not get cGVHD and are represented on the graph as scoring “0.”

FIG. 14 shows that Gut GVHD is influenced by donor APCs. (A) Percentage of weight change in WT recipients of CD80/86^{-/-} BM. Combined data from 2 experiments are shown. On day 0, WT recipient mice were lethally irradiated and reconstituted with 8×10⁶ WT BM cells alone (thin solid line; n=7), WT BM plus 2×10⁶ WT CD4 cells (bold solid line; n=17), 8×10⁶ CD80/86^{-/-} BM cells alone (thin broken line; n=6), or CD80/86^{-/-} BM plus 2×10⁶ WT CD4 cells (bold broken line; n=19). *P<0.05 or P<0.01 for CD4 recipients of WT BM versus CD80/86^{-/-} BM. (B) Percentage of weight change in WT recipients of CD40^{-/-} BM. Combined data from 2 experiments are shown. On day 0, WT recipient mice were lethally irradiated and reconstituted with 8×10⁶ WT BM cells alone (thin solid line; n=8), WT BM plus 10⁷ WT spleen cells (bold solid line; n=27), 8×10⁶ CD40^{-/-} BM cells alone (thin broken line; n=10), or CD40^{-/-} BM plus 10⁷ WT spleen cells (bold broken line; n=28). P<0.01 for spleen cell recipients of WT BM versus CD40^{-/-} BM. (C) Percentage of weight change in CD80/86^{-/-} recipients of CD40^{-/-} BM. On day 0, CD80/86^{-/-} recipient mice were lethally irradiated and reconstituted with 8×10⁶ WT BM cells alone (thin solid line; n=5), WT BM plus 2×10⁶ WT CD4 cells (bold solid line; n=16), 8×10⁶ CD40^{-/-} BM cells alone (thin broken line; n=4), or CD40^{-/-} BM plus 2×10⁶ WT CD4 cells (bold broken line; n=17). P<0.01 for CD4 recipients of WT BM versus CD40^{-/-} BM. (D) Pathology score for representative mice from panel A. Mean score is indicated by a horizontal bar. P<0.01 for WT BM+CD4 cell recipients versus all other experimental groups. P=0.19 or 0.73 for CD80/86^{-/-} BM+CD4 cells versus CD80/86^{-/-} BM control or WT BM control, respectively. (E) Pathology scores for representative mice from panel C. Mean score is indicated by a horizontal bar. P<0.01 for WT BM+CD4 cell recipients versus CD40^{-/-} BM+CD4 cell recipients.

FIG. 15 shows that TIB120-saporin depletes dendritic cells (DCs). 200 μg of TIB120-saporin was injected i.v. and cohorts of mice (3/group) were analyzed 3 and 4 days later for dendritic cell depletion from spleen and lymph node. (A), Representative flow cytometry. Dendritic cells were identified as previously described (Shlomchik et al. 1999). (B), Total dendritic cell numbers from spleen and lymph node. As controls, some mice were unmanipulated, while others received 200 μg TIB120 and unconjugated saporin.

FIG. 16 shows that TIB120-saporin depletes dendritic cells in a dose dependent fashion. Mice were injected with graded doses of TIB120-saporin and spleens were analyzed for dendritic cell content 4 days later. Shown are total number of cells; 3-4 mice/group. Note depletion of both CD11c⁺/CD8⁺ and CD11c⁺/CD8⁻ DCs. Also note that 50 μg is inferior to 100 μg.

FIG. 17 shows that TIB120-saporin adds to dendritic cell depletion induced by irradiation. Mice were injected with 50 μg of TIB120-saporin and received two 450cGy fractions of irradiation 3 days later. Dendritic cell content of spleens was analyzed 24 hours later. (A), representative flow cytometry (IT, immunotoxin; TIB120+saporin is antibody plus unconjugated saporin as a control). Plots were gated on live (not staining with propidium iodide) and lineage negative cells (not staining with Gr-1, Thy1.2, TERR119 and CD19). (B), total numbers of dendritic cells in each group (3-4 mice/group).

FIG. 18 shows that pretreatment with TIB120-saporin decreases hepatic GVHD. Mice were transplanted as described in the text. Liver histology was graded in a blinded fashion by a pathologist who specializes in liver pathology. Immunotoxin; IT. TIB120 plus unconjugated saporin; Ab+saporin. Each circle is the score of an individual animal. Dash is the mean. p<0.079 by Mann-Whitney.

FIG. 19 shows that N418-saporin depletes dendritic cells in vivo. Mice were injected i.v. with 100 μg or 50 μg of N418-saporin or 100 μg of N418 plus unconjugated saporin. Mice were sacrificed 3 days later and dendritic cells were analyzed as described above except as follows. Instead of directly staining with anti-CD11c, we visualized CD11c⁺ cells with an anti-hamster antibody. This approach relied on N418-saporin or free N418 being still bound to cells. As a control, we preincubated representative samples with N418 in vitro and then stained with an anti-hamster antibody. While this increased the intensity of staining with the anti-hamster antibody, it did not change the percentage of positive cells.

FIG. 20 shows that treatment with TIB120-saporin and N418-saporin decreases GVHD. On day-3 one group of recipient B6 mice received 5011 g of TIB120-saporin and 10011 g N418-saporin. On day 0, all mice received two 450cGy fractions, 7×10⁶ T cell depleted bone marrow from C3H.5W mice, with 0 or 2.8×10⁶ purified C3H.5W lymph node CD8⁺ T cells. Mice were then observed for the development of GVHD. A. Mice were weighed at the indicated time points; mean weights are shown (*p<0.04 comparing immunotoxin treated CD8 recipients to control CD8 recipients by unpaired t-test). B. Mice were sacrificed and tissues harvested for pathologic analysis. Shown are scores for hepatic GVHD (*p<0.006 by Mann-Whitney). Note the reduced GVHD in immunotoxin treated mice, including 3 mice with no histologic evidence of hepatic GVHD.

### DETAILED DESCRIPTION OF THE INVENTION

Graft versus host disease (GVHD), an alloimmune attack on host tissues mounted by donor T cells, is the most important toxicity of allogeneic bone marrow transplantation (alloBMT). The mechanisms by which allogeneic T cells are initially and subsequently stimulated have not been well understood. The present invention is based on the development of a MHC-identical, multiple miHA mismatched B6.C or B10.D2 (H-2^{d}) BALB/c (H-2^{d}) murine model. This model shares key features of human cGVHD. Its dominant features include skin fibrosis as a result of increased collagen deposition, follicular dropout, loss of subdermal fat, and dermal mononuclear infiltrates. Hepatic disease is characterized by intrahepatic and extrahepatic bile duct mononuclear infiltration followed by fibrous thickening and sclerosis of the bile duct wall (Li et al. 1996; Nonomura et al. 1993; Vierling et al. 1989). Pulmonary fibrosis has been observed (McCormick et al. 1999) as has inflammation and destruction of salivary and lacrimal glands.

In this model, it was found that donor APCs function in CD8-mediated GVHD and either host or donor APCs were sufficient to induce murine cGVHD. These results suggest that strategies that target either donor- or host-derived APCs may mitigate the manifestations of CD4-dependent GVHD and/or cGVHD and provide a strong rationale for targeting both donor and host APCs, rather than just host APCs.

The data presented herein establishes that GVHD, in particular, cGVHD, resulted from immune reactivity to grafts and transplants involves both donor and host APCs and that immune reactivity can be prevented and/or treated by depletion and/or inhibition/inactivation of both donor and host APCs. Without wishing to be bound by theory, it is believed that it is the T cells in the donor population which are responsible for the graft versus host disease.

Thus, the invention includes methods of depleting host and donor APCs in preventing, ameliorating, decreasing, and/or treating GVHD. The methods comprise contacting a population of hematopoietic cells in the mammal with an antigen presenting cell depleting and/or inhibiting/inactivating composition to effect depletion and/or inhibition/inactivation of antigen presenting cells in the population of hematopoietic cells. As used herein, “Antigen Presenting Cells” or “APC's” include known APCs such as Langerhans cells, veiled cells of afferent lymphatics, dendritic cells and interdigitating cells of lyphods organs. The definition also includes mononuclear cells such as lymphocytes and macrophages.

Depletion and/or inhibition/inactivation of donor and host APCs is beneficial to a host having a pathological immune response. Methods of depleting or inhibiting a particular cell population are well known to those of ordinary skill in the art. In one aspect, depletion and/or inhibition/inactivation of APCs may be accomplished by contacting a population of hematopoietic cells containing APCs with an antigen presenting cell depleting and/or inhibiting/inactivating composition. The depletion and/or inhibition of APCs may be carried out in vitro or in vivo in the mammal. According to the present invention, the depletion and/or inhibition/inactivation of APCs in the donor and/or host may be carried out before, at the time of, or after the transplantation.

In one embodiment, depletion and/or inhibition of either host or donor APCs alone or both host and donor APCs is conducted after the administration of donor hematopoietic cells or transplants to the host. Depletion and/or inhibition/inactivation of the APCs prior to GVHD can prevent GVHD, while depletion and/or inhibition/inactivation of the APCs after GVHD can be used to treat GVHD. The present invention also contemplates both preventing and treating GVHD for a single transplantation event or for multiple transplantation events.

Depletion of either donor or host APCs or both donor and host APCs can begin at any time after transplantation (e.g., within minutes, hours, or 1, 2, 4, 6, 8, 10, or 30 days after donor hematopoietic cells or transplants are introduced into the recipient). Post transplantation depletion and/or inhibition/inactivation of APC may also be provided at least two months or six months after the previous administration of hematopoietic cells and transplants; at least 1, 2, 4, 6, 8, 10, or 30 days, two months, six months, or at any time in the life span of the recipient after the transplantation of a transplant; when the recipient begins to show signs of rejection or GVHD, e.g., as evidenced by a decline in function of the grafted organ, by a change in the host donor specific antibody response, or by a change in the host lymphocyte response to donor antigen; when the level of chimerism decreases; when the level of chimerism falls below a predetermined value; when the level of chimerism reaches or falls below a level where staining with a monoclonal antibody specific for a donor PBMC antigen is equal to or falls below staining with an isotype control which does not bind to PBMC's or generally, as is needed to maintain tolerance or otherwise prolong the acceptance of a transplant.

In another embodiment, depletion and/or inhibition/inactivation of either host or donor APCs alone or both host and donor APCs may be carried out simultaneously with the administration of donor hematopoietic cells or transplants. Depletion and/or inhibition/inactivation of either host APCs or both donor and host APCs at the time of transplantation or anytime afterwards can have an effect on later complications like cGVHD.

Alternatively, either host or donor APCs alone or both host and donor APCs are depleted and/or inhibited/inactivated prior to the administration to the host organism of donor hematopoietic cells. Then, APCs in the host can also be depleted and/or inactivated/inhibited at any time(s) subsequent to the transplantation (e.g., after onset of GVHD).

Thus, the present invention may be practiced with various steps of depleting and/or inhibiting/inactivating APCs and introducing donor hematopoietic cells or transplants. In one embodiment, the invention provides methods of preventing, ameliorating, decreasing, and/or treating graft versus host disease (GVHD) in a host mammal by (a) transferring hematopoietic cells from a donor mammal to said host mammal; (b) depleting and/or inhibiting/inactivating antigen presenting cells in a population of hematopoietic cells in said host mammal, wherein the antigen presenting cells are depleted and/or inhibited/inactivated by an antigen presenting cell depleting and/or inhibiting/inactivating composition. Irradiation of the host may be used in conjunction with the administration of the composition, wherein the irradiation can be administered before, during or after administration of the APC depleting and/or inhibiting/inactivating composition.

In another embodiment, the methods of the present invention involve the steps of (a) depleting and/or inhibiting/inactivating antigen presenting cells in a population of hematopoietic cells in said host mammal, (b) depleting and/or inhibiting/inactivating antigen presenting cells in a population of hematopoietic cells from a donor mammal; and (c) transplanting the donor hematopoietic cells to said host mammal, wherein said graft versus host disease is prevented or treated in said host mammal by virtue of said depletion and/or inhibition/inactivation of said host and donor antigen presenting cells, wherein the antigen presenting cells are depleted by an antigen presenting cell depleting and/or inhibiting/inactivating composition.

In some embodiments, the present invention provides a method of preventing or treating GVHD by (a) depleting and/or inhibiting/inactivating antigen presenting cells in a population of hematopoietic cells in said host mammal; (b) transplanting donor hematopoietic cells from a donor mammal to said host mammal; and (c) depleting and/or inhibiting/inactivating the antigen presenting cells in a population of hematopoietic cells from a donor mammal, wherein the antigen presenting cells are depleted and/or inhibited/inactivated by an antigen presenting cell depleting and/or inhibiting/inactivating composition.

In still some other embodiments, the present invention provides a method of preventing or treating GVHD by (a) depleting and/or inhibiting/inactivating antigen presenting cells in a population of hematopoietic cells from a donor mammal; (b) transplanting the donor hematopoietic cells to said host mammal; and (c) depleting and/or inhibiting/inactivating antigen presenting cells in a population of hematopoietic cells in said host mammal, wherein the antigen presenting cells are depleted and/or inhibited/inactivated by an antigen presenting cell depleting composition.

The GVHD to be treated or prevented includes, but is not limited to, acute GVHD, refractory GVHD, chronic GVHD, CD4-mediated GVHD, or CD8-mediated GVHD. The term “graft versus host disease” or “GVHD” as used herein is the pathological reaction that occurs between the host and grafted tissue. The grafted or donor tissue dominates the pathological reaction. Graft versus host disease (GVHD) can be seen following stem cell and/or solid organ transplantation. GVHD occurs in immunocompromised subjects, who when transplanted, receive “passenger” lymphocytes in the transplanted stem cells or solid organ. These lymphocytes recognize the recipient's tissue as foreign. Thus, they attack and mount an inflammatory and destructive response in the recipient. The disease includes acute and chronic GVHD. Acute GVHD (aGVHD) usually occurs within the first three months following a transplant, and can affect the skin, liver, stomach, and/or intestines. Chronic GVHD (cGVHD) is the late form of the disease, and usually develops three months or more after a transplant. The symptoms of chronic GVHD resemble spontaneously occurring autoimmune disorders such as lupus or scleroderma.

The term “recipient” or “host” as used herein refers to any subject that receives an organ and/or tissue transplant or graft.

“Transplant,” as used herein, refers to a body part, organ, tissue, or cells. Organs such as liver, kidney, heart or lung, or other body parts, such as bone or skeletal matrix, tissue, such as skin, intestines, endocrine glands, or progenitor stem cells of various types, are all examples of transplants.

By the term “APC depleting composition” as used herein, is meant a composition which when contacted with an APC is capable of killing the APC or is capable of incapacitating the APC such that the APC is non-functional.

The terms “impairment of APC function” or “non-functional APC” are used essentially interchangeably herein and include an APC which is incapable of stimulating T cells in an antigen specific fashion. Thus, methods which impair APC function include any method which prevents an APC from stimulating T cells in an antigen specific fashion. Such methods include, but are not limited to, depleting APCs in a host and preventing APC costimulatory function. For example, antibodies, such as for example, antibodies directed against key costimulatory molecules such as B71 and/or B72 may be used to impair APC costimulatory function.

Depletion of APCs in an animal may be accomplished in any number of ways. For example, APC depletion may be accomplished using an immunotoxin conjugated to an antibody. The immunotoxin is a molecule which is capable of killing an APC, and may include, but not be limited to ricin, diptheria toxin, pseudomonas exotoxin A, ribosome inhibitory proteins, radioactivity, radiolabeled antibodies, or any other heretofore unknown or known toxin. Examples of suitable toxins and the methods of generating the same can be found in the following list of references. (Levy et al., 1991, J. Clin. Oncol. 9:537-538; Burbage et al., 1997, Leukemia Res. 21:681690; Chandler et al., 1996, Seminars in Pediatric Surgery 5:206-211; Collinson et al., 1994, J Immunopharmacology 16:37-49; Essand et al., 1998, Internatl. J. Cancer 77:123-127; Faguet et al., 1997, Leukemia & Lymphoma 25:509-520; Flavell et al., 1995, Brit. J. Cancer 72:1373-1379 (describing the production and use of saporin-antibody immunotoxin conjugates on page 1374), the contents of which are specifically incorporated by reference in its entirety herein; Frankel et al., 1997, Leukemia & Lymphoma 26:287-298; Knowles et al., 1987, Anal. Biochem. 160:440-443; Kreitman et al., 1997, Blood 90:252-259; Lynch et al., 1997, J. Clin. Oncol. 15:723-734; Mansfield et al., 1997, Blood 90:2020-2026; Maurer-Gebhard et al., 1998, Cancer. Res. 58:2661-2666; O'Toole et al., 1998, Curr. Topics in Microbiol. & Immunol. 234:35-56; Press et al., 1998, Cancer Journal From Scientific American 4:S19-S26; Przepiorka et al., 1995, Bone Marrow Transplantation 16:737-741; Schnell et al., 1996, Internatl. J. Cancer 66:526-531; Spyridonidis et al., 1998, Blood 91:1820-1827; Winkler et al., 1997, Annals of Oncol. 8:139-146; Kuzel et al., 1993, Leukemia & Lymphoma 11:369-377; Moreland et al., 1995, Arthritis & Rheumatism 38:1177-1186; LeMaistre et al., 1993, Cancer Res. 53:3930-3934), each of which are specifically incorporated by reference in their entirety.

Toxins may be generated using recombinant DNA methodology, or they may be obtained biochemically. When the toxin is obtained using recombinant DNA methodology, DNA encoding the toxin is cloned into a suitable vector, the vector is transfected into a suitable host cell and the toxin is generated in the host cell following transcription and translation of the DNA. Preferably, for the purposes of the present invention, DNA encoding the toxin is cloned in frame with DNA encoding a receptor or an antibody, which receptor or antibody is specific for a molecule expressed by an APC. Thus, the chimeric toxin molecule so generated is specific for an APC, targets the APC, binds thereto, and in some manner, effects impairment of or kills the APC.

Examples of toxins which are conjugated to an antibody or receptor molecule include the Pseudomonas A toxin. While the invention should in no way be construed to be limited to the use of this particular toxin, examples of chimeric molecules which include this toxin are provided in the following references to exemplify one embodiment of the invention. (Essand et al., 1998, Internatl. J. Cancer 77:123-127; Kreitman et al., 1997, Blood 90:252-259; Mansfield et al., 1997, Blood 90:2020-2026; Maurer-Gebhard et al., 1998, Cancer Res. 58:2661-2666; Spyridonidis et al., 1998, Blood 91:1820-1827; Bera et al., 1998, Molecular Medicine 4:384-391; Francisco et al., 1998, Leukemia & Lymphoma 30:237-245; Kreitman et al., 1998, Advanced Drug Delivery Reviews 31:53-88; Wu, 1997, Brit. J. Cancer 75:1347-1355; Zdanovsky et al., 1997, Faseb Journal 11:A1325-A1325).

By the term “immunotoxin” as used herein, is meant a compound which when in contact with an APC, is capable of killing the APC or incapacitating the APC such that the APC is non-functional.

In order to deplete APCs, the immunotoxin is preferably conjugated to an antibody, which antibody is specific for an epitope on an APC. Thus, the immunotoxin is directed to the APC by virtue of the antibody conjugated thereto. Epitopes which may be targeted on an APC include, but are not limited to, CD1a, CD11c, MHCII, CD11b, and DEC205. Additional epitopes include B71, B72, CD40, and Type I and Type II lectins. These are particularly attractive candidates as they can have cytoplasmic domains that signal for endocytosis when the receptor is engaged. Also included are matrix metalloproteins such as decysin, and chemokine receptors.

The-antibody which is used may also be radioactively labeled, preferably, labeled with radioactive iodine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of protein molecules which faun an immunoglobulin molecule. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1988, Antibodies: A Laboratory Manual, Cold Spring Harbor, N.Y. Houston et al., 1988, Proc. Natl. Acad. Sci. USA 85:5879-5883; Bird et al., 1988, Science 242:423-426).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described; for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. in Immunol. 12(3,4): 125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., spleen cells or a hybridoma, which cells express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encodes the desired antibody, may be engineered such that the protein is displayed on the surface thereof in such a manner that it is available for binding to its corresponding binding protein, e.g., the antigen against which the antibody is directed. Thus, when bacteriophage which expresses a specific antibody is incubated in the presence of a cell which expresses the corresponding antigen, the bacteriophage will bind to the cell. Bacteriophage which does not express the antibody will not bind to the cell. Such panning techniques are well known in the art and are described for example, in Wright et al., (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phages which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

APC depletion may also be accomplished by selectively introducing a gene into the APC, the expression of which gene either directly results in APC cell death or renders the APC specifically susceptible to other pharmacological agents. In vivo or ex vivo depletion of APCs according to this method may be accomplished by delivering the desired gene to the APC using a viral gene delivery systems such as, but not limited to a retrovirus, adenovirus or an adeno-associated virus gene delivery system. The desired viral delivery system may comprise a virus whose genome encodes a protein which, for example, directly causes cell death, for example by inducing apoptosis of the APC. Alternatively, the viral delivery system may contain a virus whose genome encodes, for example, a herpes simplex virus thymidine kinase gene. Expression of the herpes simplex virus thymidine kinase gene in the APC renders the APC sensitive to pharmacologic doses of ganciclovir. Thus, subsequent contact of the virally transduced APC with ganciclovir results in death of the APC. Such gene transfer approaches may be used in an ex vivo method of transducing human bone marrow, followed by infusion of bone marrow so transduced into the patient. These patients would then be treated with ganciclovir and then undergo a second therapeutic transplant of bone marrow in a manner similar to that described in the experimental examples presented herein.

Agents such as ganciclovir which mediate killing of a cell upon expression of a gene such as thymidine kinase, are referred to herein as “corresponding agents.” Hematopoietic stem cells can be collected from the patient by collecting aspirations from the iliac crest. This is performed under general anesthesia if large numbers are needed. More commonly, hematopoietic cells are obtained from the peripheral blood of the patient via leukopheresis. Leukopheresed patients may be pretreated with either chemotherapy or with hematopoietic growth factors such as GCSF and GMCSF in order to increase the numbers of circulating progenitor cells.

Genes which can be used to kill APCs include, but are not limited to, herpes simplex virus thymidine kinase and cytosine deaminase, or any gene which induces the death of a cell that can be placed under the control of an inducible promoter/regulatory sequence (referred to interchangeably herein as a “promoter/regulatory sequence” or as a “promoter”). The gene is transferred into a patient's primitive hematapoietic cells, the cells are selected under an appropriate selective pressure, the cells are transferred to the patient, and are allowed to engraft therein. The patient is then treated with an agent which induces promoter activity, thereby inducing expression of the gene whose product functions to kill APCs. In the case of thymidine kinase, other agents which facilitate killing of the cell by this enzyme may also be used, such as, for example, ganciclovir (Bonin et al., 1997, Science 276:1719-1724; Bordignon et al., 1995, Human Gene Therapy 6:813-819; Minasi et al., 1993, J. Exp. Med. 177:1451-1459; Braun et al., 1990, Biology of Reproduction 43:684-693). Other genes useful for this purpose include, but are not limited to, constitutively active forms of caspases 3, 8, and 9, bax, granzyme, diphtheria toxin, Pseudomonas A toxin, ricin and other toxin genes are disclosed elsewhere herein. The generation of appropriate constructs for delivery of such genes to a human will be readily apparent to the skilled artisan and is described, for example, in Sambrook et al. (1989, Molecular Cloning: A Laboratory Manual, Cold Spring Harbor Laboratory, New York) and in Ausubel et al. (1997, Current Protocols in Molecular Biology, John Wiley & Sons, New York).

It is important that the gene which is transferred into the cells, for the purpose of killing the cells, be placed under the control of the appropriate promoter sequence, such that induction of expression of the gene may be effected upon addition to the cells (administration to the mammal) of the appropriate inducer. Such inducible promoter sequences include, but are not limited to promoters which are induced upon addition of a metal to the cells, steroid inducible promoters and the like. In one preferred embodiment, the ecdysone promoter system may be employed. In this embodiment, the ecdysone promoter is cloned upstream of the ecdysone receptor protein sequence, which is positioned upstream of a second promoter sequence which drives expression of the ecdysone binding site operably linked to the desired gene, for example, the desired toxin. Induction of the promoter induces expression of the toxin, thereby effecting killing of the cell in which the toxin gene resides.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The use of viral and non-viral vectors for delivery of genes to hematapoietic cells is contemplated in the invention. Viral vectors include, but are not limited to, retroviral, adenoviral, herpesviral and other viral vectors which are well known in the art and are described, for example, in Sambrook et al. (1989, Molecular Cloning: A Laboratory Manual, Cold Spring Harbor Laboratory, New York) and in Ausubel et al. (1997, Current Protocols in Molecular Biology, John Wiley & Sons, New York). It is important of course, that any viral vector delivery system used employ a virus which is replication incompetent. As stated, non-viral vectors such as liposomes and the like, may also be used to deliver an APC depleting or inhibiting composition to a human.

Cells which have transduced therein a gene for cell killing, when such cells are transduced in an ex vivo manner, may be selected (i.e., separated from cells which do not comprise the gene) by providing the cells with a selectable marker in addition to the transduced gene. Selectable markers are well know in the art and are described, for example, in Sambrook et al. (1989, Molecular Cloning: A Laboratory Manual, Cold Spring Harbor, N.Y.).

APC depletion may further be accomplished by introducing into a population of APCs an oligonucleotide (for example, but not limited to, an antisense molecule) or a ribozyme, which oligonucleotide or ribozyme is capable of inducing death of the APC, or of inducing impairment of APC function. Such oligonucleotides include those which target an essential function of an APC, defined herein as being one which either kills an APC or impairs the function of the APC with respect to stimulation of T cells. Such functions of an APC include, but are not limited to, the costimulatory function of B71 and B72, CD40, among others. Thus, oligonucleotides and ribozymes which are useful in the methods of the invention include, but are not limited to, those which are directed against these targets.

Also included are oligonucleotides which contain at least one phosphorothioate modification are known to confer upon the oligonucleotide enhanced resistance to nucleases. Specific examples of modified oligonucleotides include those which contain phosphorothioate, phosphotriester, methyl phosphonate, short chain alkyl or cycloalkyl intersugar linkages, or short chain heteroatomic or heterocyclic intersugar (“backbone”) linkages. In addition, oligonucleotides having morpholino backbone structures (U.S. Pat. No. 5,034,506) or polyamide backbone structures (Nielsen et al., 1991, Science 254: 1497) may also be used. The examples of oligonucleotide modifications described herein are not exhaustive and it is understood that the invention includes additional modifications of the antisense oligonucleotides of the invention which modifications serve to enhance the therapeutic properties of the antisense oligonucleotide without appreciable alteration of the basic sequence of the antisense oligonucleotide.

As used herein, the term “antisense oligonucleotide” means a nucleic acid polymer, at least a portion of which is complementary to a nucleic acid which is present in a normal cell. The antisense oligonucleotides of the invention preferably comprise between about fourteen and about fifty nucleotides. More preferably, the antisense oligonucleotides comprise between about twelve and about thirty nucleotides. Most preferably, the antisense oligonucleotides comprise between about sixteen and about twenty-one nucleotides.

As noted herein, depletion of APC includes impairment of APC function. Impairment of APC function includes all forms of APC impairment with or without physical removal or depletion of APCs. Thus, impairment of APC function includes the use of an antibody that blocks the function of APC surface molecules which are critical for APC function. Such APC surface molecules include, but are not limited to B71, B72 and DEC205. Antibodies directed against B71, B72 and CD40 are available from Pharmingen, San Diego, Calif. Anti-DEC205 antibodies and anti-MHC-II antibodies are available from Pharmingen and from the American Type Culture Collection.

Alternatively, peptides which block the function of APC surface molecules, which blocking results in impairment of APC function, may be used to effectively deplete APCs in a host organism. Such peptides include, but are not limited to, those which are designed to specifically bind receptor molecules on the surface of APCs, and those which are designed to, for example, inhibit essential enzymatic functions in these cells.

Similarly, genes and oligonucleotides which are designed for the same purpose as described herein, are also included as tools in the methods of the invention. Thus, peptides, oligonucleotides and genes which impair the biological function of an APC, as that term is defined herein, are also contemplated for use in the methods of the invention disclosed herein.

APC “depletion or impairment” as used herein, should be construed to include depletion of sufficient antigen presenting cells prior to or concurrent with allogeneic bone marrow transplantation, including, but not limited to dendritic cells, B lymphocytes and macrophages to prevent graft versus host disease in the patient. The term should also be construed to include selective depletion of macrophages, selective depletion of dendritic cells, functional impairment of all antigen presenting cells including, but not limited to dendritic cells, macrophages, and B cells, selective functional impairment of macrophages, and selective functional impairment of dendritic cells.

The invention thus also includes a method of preventing or treating graft versus host disease, in particular chronic GVHD, in a mammal. The method comprises contacting a population of hematopoietic cells, in particular donor hematopoietic cells, in the mammal with an antigen presenting cell depleting or inhibiting composition to effect depletion of antigen presenting cells in the population of hematopoietic cells, and transferring donor hematopoietic cells to the mammal, wherein the graft versus host disease is prevented in the mammal by virtue of the depletion of the antigen presenting cells. The population of hematopoietic cells may be contacted with the antigen presenting cell depleting or inhibiting composition in vivo in the mammal. The preferred mammal is a human.

The invention also includes a method of preventing or treating graft versus host disease, in particular chronic GVHD, in a mammal. The method comprises obtaining a population of hematopoietic stem cells, in particular donor hematopoietic cells, from the mammal. A gene is added to the cells which when expressed in the cells is capable of killing the cells. Cells which have received the gene are selected by virtue of the fact that the cells are co-transfected with a selectable marker. The mammal is irradiated to remove bone marrow cells in the mammal. The selected cells are added to the mammal and expression of the gene in the selected cells is induced thereby effecting killing of antigen presenting cells in the mammal. The mammal is then provided with an allogeneic bone marrow transplant, wherein graft versus host disease is prevented in the mammal by virtue of the killing of the antigen presenting.

In a preferred embodiment, the gene is operably linked to an inducible promoter and expression of the gene is effected by administration of an inducer of the promoter to the mammal. In another preferred embodiment, the gene encodes a toxin.

Also included is a method of preventing graft versus host disease in a mammal This method comprises obtaining a population of hematopoietic stem cells from the mammal, adding to the cells a gene which when expressed in the cells in the presence of a corresponding agent is capable of killing the cells. Cells having the gene are selected. The mammal is irradiated to remove bone marrow cells in the mammal. The selected cells are added to the mammal, and the corresponding agent is also added to the mammal to effect killing of the selected cells in the mammal thereby effecting killing of antigen presenting cells in the mammal. The mammal is provided with an allogeneic bone marrow transplant, wherein graft versus host disease is prevented in the mammal by virtue of the killing of the antigen presenting cells.

In a preferred embodiment, the gene is thymidine kinase, the gene is operably linked to a constitutive promoter and the corresponding agent is ganciclovir. In another preferred embodiment, the gene is thymidine kinase, the gene is operably linked to an inducible promoter, the corresponding agent is ganciclovir, and prior to adding the corresponding agent to the mammal, the expression of the gene is induced by administration of an inducer of the promoter to the mammal.

“Prevention of graft versus host disease” in a mammal, as the term is used herein, means reducing the severity of the graft versus host disease which would occur in the absence of any treatment, or ablating graft versus host disease as a result of the treatment.

The type of immunosuppression aimed at APCs which is disclosed herein may be used to prevent GVHD completely or partially in any situation in which allogeneic bone marrow transplantation might be performed. Such situations include, but are not limited to the following: Hematologic malignancies, such as, but not limited to, acute myeloid leukemia, acute lymphoid leukemia, chronic myelogenous leukemia, lymphomas, chronic lymphocytic leukemia, myelodysplasia and preleukemias, multiple myeloma, essential thrombocythemia, myelofibrosis, polycythemia vera, and paroxysmal nocturnal hemaglobinuria. Autoimmune cytopenias, including, but not limited to aplastic anemia, amegakaryocytic thrombocytopenia, immune thrombocytopenia, autoimmune hemolytic anemia, and autoimmune neutropenias. Genetic disorders including, but not limited to hemaglobinopathies such as sickle cell disease and thalasemias, severe combined immune deficiency disorders, such as adenosine deaminase deficiency and lysosomal storage diseases, such as Gaucher's Disease. Other autoimmune diseases including, but not limited to rheumatoid arthritis, systemic lupus erythematosis, Sjogren's syndrome, multiple sclerosis, vasculitides, dermatomyosisitis, polymyositis, and ankylosing spondylitis. Also included is solid organ transplantation. Further, the methods of the invention are useful as immunosuppressive therapy in the absence of allogeneic bone marrow transplantation. Because depletion of functional antigen presenting cells is effective in preventing GVHD, one of the most potent in vivo T cell stimuli, it is likely to also be effective outside of the allogeneic bone marrow transplant context as therapy for any of the autoimmune cytopenias or autoimmune diseases disclosed herein.

The invention further encompasses the use pharmaceutical compositions of an appropriate APC depleting composition to practice the methods of the invention, the compositions comprising an appropriate APC depleting composition and a pharmaceutically-acceptable carrier.

As used herein, the term “pharmaceutically-acceptable carrier” means a chemical composition with which an appropriate APC depleting composition may be combined and which, following the combination, can be used to administer the appropriate APC depleting composition to a mammal.

The pharmaceutical compositions useful for practicing the invention may be administered to deliver a dose of between 1 ng/kg/day and 100 mg/kg/day.

Pharmaceutical compositions that are useful in the methods of the invention may be administered systemically in oral solid formulations, ophthalmic, suppository, aerosol, topical or other similar formulations. In addition to the APC depleting composition, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate APC depleting composition according to the methods of the invention.

The invention is now described with reference to the following experimental examples. These examples are provided for the purpose of illustration only and the invention should in no way be construed as being limited to these examples but rather should be construed to encompass any and all variations which become evident as a result of the teaching provided herein.

### EXAMPLES

### Example 1

### Prevention of GVHD by Selective Inactivation of Host APCs

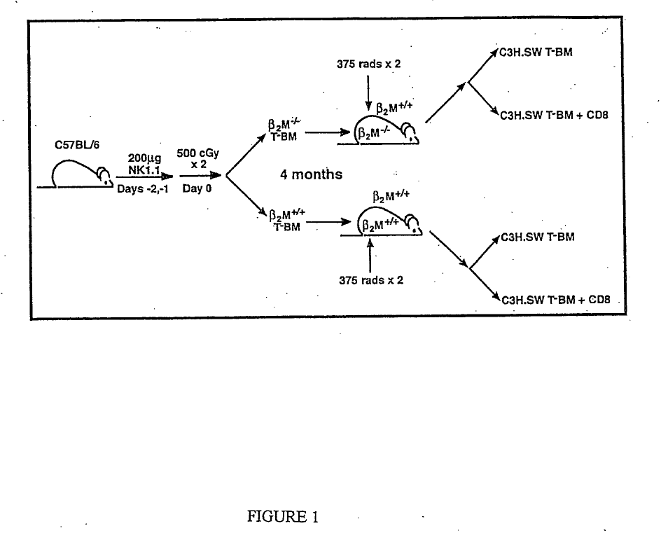

In order to address whether donor or host APCs initiate GVHD, a genetic approach was taken to ask whether host mice whose APCs' were incapable of presenting MHC I restricted peptides would support a GVHD reaction. First, mice were generated that did not express MHC I on their APCs but did express MHC I on target tissues. Such mice were constructed as bone marrow chimeras (FIG. 1) using wild type C57BL/6 (B6; H-2^{b}) hosts and B6 β-2-microglobulin knock out mice (β₂M^{-/-}) as bone marrow donors (β₂M−/−→B6) chimeras) (Koller et al., 1990, Science 248:1227-1230). Because β₂⁻)-microgloblulin is part of the MHC I complex, cells obtained from these mice do not express MHC I and therefore cannot present peptide antigens to CD8⁺ T cells (Koller et al., 1990, Science 248:1227-1230). After waiting four months to allow for (β₂M^{-/-}) bone marrow engraftment and APC repopulation, these chimeras were used as recipients in a second allogeneic bone marrow transplant designed to cause GVHD. This was performed essentially as follows. Bone marrow was flushed from femurs and tibias with DMEM plus 10% fetal bovine serum. Red cells were lysed using the NH₄Cl/Tris method, washed and resuspended in PBS with 0.5% BSA and 5 mm EDTA. BM cells were T cell depleted with anti-Thy 1.2 labeled microbeads (Miltenyi Biotech, Auburn, Calif.) according to the manufacturer's protocol. T cell depletion was confirmed by staining with a combination of CD4 FITC, CD8 FITC and CD3 PE labeled antibodies (Pharmingen, San Diego, Calif.), clones RM4-5, 53-6.7 and 500-A2, respectively. After exclusion of dead cells by propidium iodide staining, residual T cells were between 0.01-0.06% of total live cells. Thus, T cell depleted C3H.SW (H-2^{b}) bone marrow (C3H.SW T⁻BM) with or without 10⁶ or 2×10⁶ highly purified C3H.SW CD8+ T cells (FIG. 1) was infused into the previously generated chimeric recipients following a second dose of radiation. As controls, B6 recipients received syngeneic marrow in the first transplant (B6→136), and were then treated identically as the β₂M^{-/-}→B6 chimeras.

Highly purified C3H.SW CD8⁺ T cells were obtained as follows. CD8⁺ T cells were isolated by negative depletion of C3H.SW lymph nodes by first staining cells with biotin labeled antibodies against CD4 and CD45R (B220) (clones GK. 1.5 and RA3-6B2), and CD11b (clone M1/70) (Pharmingen, San Diego, Calif.), followed by the addition of streptavidin conjugated magnetic beads (Miltenyi Biotech, Auburn Calif.). Negative depletion was performed according to the manufacturer's protocol. To confirm the purity of the CD8⁺ T cells, cells were stained with antibodies against CD4, CD8, CD11b, and CD45R (B220). Ninety five percent of the cells were CD8+; CD4+ or CD3+/CD8-cells were <0.25%.

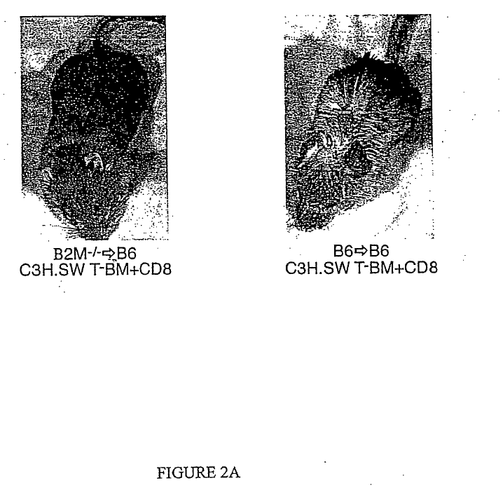

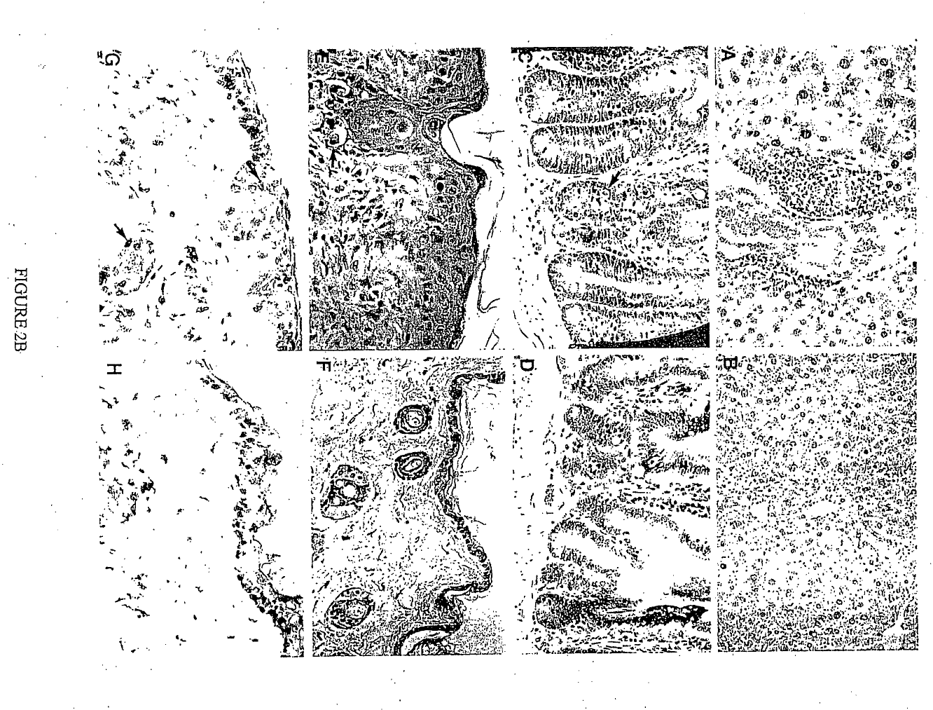

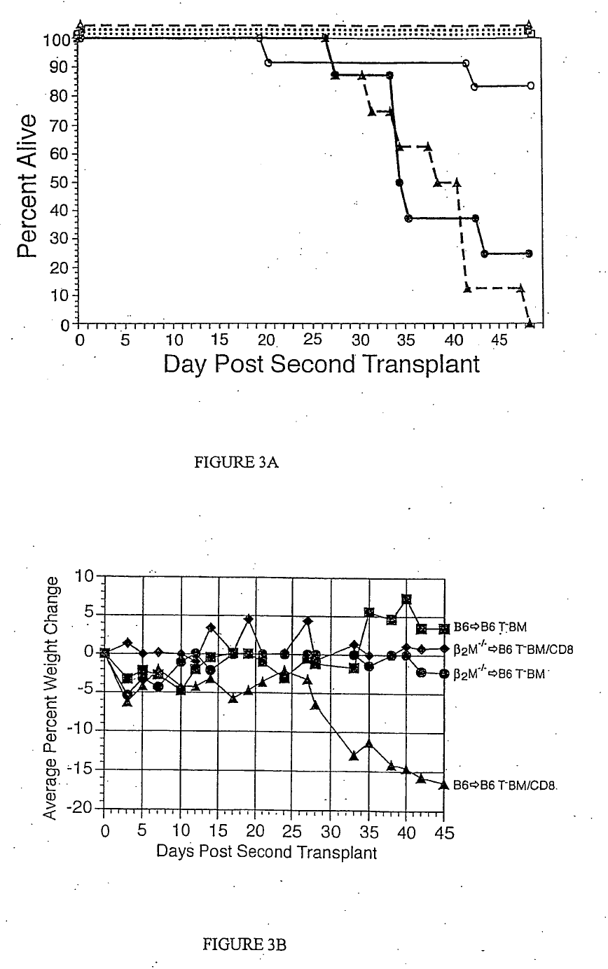

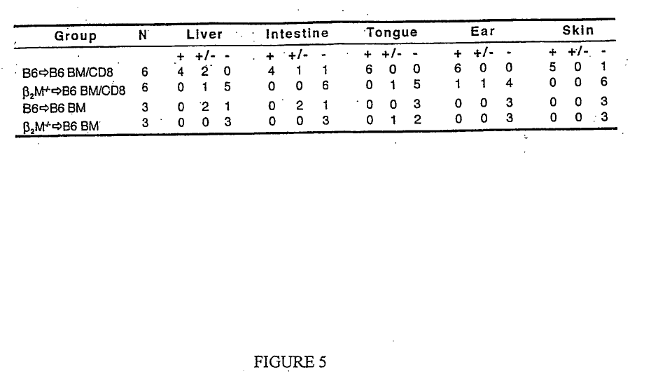

Strikingly, in each of three experiments, the β₂M^{-/-}→B6 recipients of BM plus CD8⁺ T cells were resistant to the induction of acute GVHD (FIG. 2A, left panel). On the other hand, as expected, the B6→B6 recipients of C3H.SW BM plus CD8 cells developed severe acute GVHD manifested by hunched posture, erythema of ears and skin, alopecia (FIG. 2A, right panel), weight loss (FIG. 3A), and death. In the first experiment (experiment 1), mice were sacrificed for histologic analysis of liver, back skin, ears, tongue and small bowel and these tissues were examined for pathologic evidence of GVHD. Blinded readings of this pathology are summarized in the Table in FIG. 5. This experiment was performed as follows. Formalin fixed, paraffin embedded sections were stained with hematoxylin and eosin. Slides were coded and were examined blindly by pathologists expert in either gastrointestinal or cutaneous pathology. Small intestine sections were evaluated for overall architectural integrity, degree and type of inflammation in the lamina propria, and epithelial injury. The assessment of epithelial injury consisted of a subjective grading of the number of apoptotic cells within epithelial crypts on a scale of 1-3+ and the degree of mucosal inflammation, both intraepithelial and lamina propria. For each animal, the degree of activity in each of these areas was then combined to give an overall interpretation of positive, indefinite, or negative for GVHD. Positive animals had an apoptosis score of at least 2+, and had increases in intra-epithelial and lamina propria inflammatory cells that were evident at low to medium power. Indefinite animals had 2+ apoptosis, but no increase in inflammation or other evidence of injury. Negative animals had up to 1+ apoptosis and no other abnormalities. Liver sections were evaluated for the presence and degree (1-3+) of portal inflammatory infiltrates, endothelialitis (portal or central), cholangiolitis, and lobar necroinflammatory changes. An overall interpretation of positive, indefinite or negative for GVHD was given for each animal. Animals received a positive score if mixed inflammatory infiltrates with endothelialitis and/or cholangiolitis were present in any portal tract. Indefinite animals had occasional portal or central lymphocytic infiltrates that lacked other inflammatory cells and lacked cholangiolitis/endothelialitis. Negative animals had essentially no infiltrates, no cholangiolitis and no endothelialitis. Tongue, skin and ear biopsies were graded according to the presence of mononuclear cell infiltrates, interface changes, dermal fibrosis, and number of apoptotic cells per linear millimeter. Positive cells had to be at least 1+ in more than one criterion. Negative animals had up to 1+ apoptosis and no other abnormalities. Indeterminate animals were 1+ in only one criterion except apoptosis.

Of 30 tissues examined from β₂M^{-/-} →B6 CD8 recipients, only one ear biopsy was read as having GVHD. in contrast, 26/30 tissues from B6→B6 CD8 recipients were read as clearly demonstrating GVHD pathology. Representative histologic sections are shown in FIG. 2B. Immunohistochemical staining of skin obtained from B6→B6 CD8 recipients demonstrated CD8⁺ cells infiltrating the epidermis whereas no CD4⁺ T cells were seen in this site, confirming the pathogenic role of CD8⁺ T cells in this model (FIG. 2B; panels G, H).

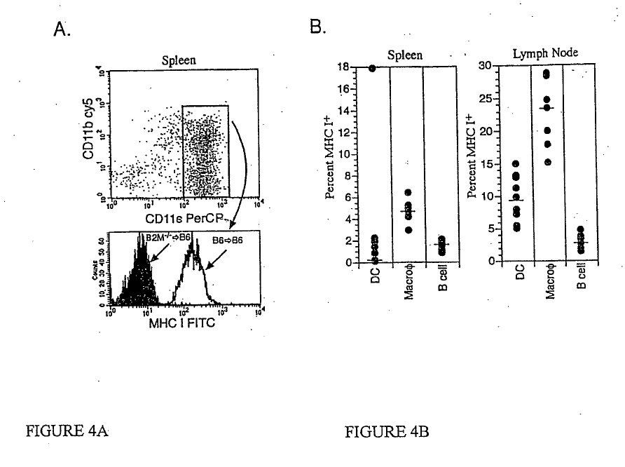

In a separate experiment (experiment 2) β₂M^{-/-}→B6 and B6→B6 chimeras received 0, 1×10⁶ or 2×10⁶ C3H.SW CD8 cells and C3H.SW T⁻BM, and were followed for survival rather than being sacrificed for pathologic analysis (FIG. 4). Again, GVHD was markedly inhibited or absent in mice with β₂M^{-/-}) BM. Six of eight and 8/8 B6→B6 recipients of 1×10⁶ and 2×10⁶ C3H.SW CD8 cells died with clinical GVHD, whereas only two deaths occurred in the 24 β₂M^{-/-→}B6 chimeric recipients of C3H.SW CD8 cells (p⁼0.0024, Fisher's exact test; comparison between all B6→B6 and β₂M^{-/-}→B6 CD8⁺ T cell recipients). It is unlikely that the β₂M^{-/-}→B6 BM/1×10⁶ death 10 days post the second transplant was due to GVHD as it occurred 15 days prior to the earliest onset of GVHD lethality seen in this system. No clinical GVHD developed in any of the β₂M^{-/-}→B6×10⁶ CD8 recipients.

In a third similar experiment in which all mice received 2×10⁶ CD8 T cells, GVHD was again inhibited in the β₂M^{-/-}→B6 CD8 cell recipients. However, in this case, some clinical GVHD was observed among 3 of 8 mice in this group (compared to 7 of 8 positive controls), although this GVHD was delayed (40% longer mean time to onset) and was less severe (46% less average weight loss) than was observed in the B6→B6 CD8 cell recipients.

The finding of milder and delayed “breakthrough” GVHD among 3 of 38 β₂M^{-/-}→B6 CD8 cell recipients over three experiments suggested that replacement of host MHC I⁺APCs with β₂M^{-/-} MHC I⁻APCs might be somewhat variable and incomplete. Ideally, in the β₂M^{-/-}→B6 chimeras, 100% of BM-derived APC, including DC, macrophages, and B cells, would be MHC I negative. To determine the degree to which this was actually achieved, flow cytometry analysis was performed on spleen and lymph node cells from a cohort of β₂M^{-/-}→B6 chimeras within one week of the second allogeneic transplant (Experiment 2; FIG. 4). Dendritic cells were identified using four color flow cytometry. Although most of the APC's were indeed MHC I negative, in every case there were residual MHC I+cells. 11/12 mice had less than 3% residual MHC I⁺ splenic dendritic cells; 1 chimera had 17.8% MHC I⁺ splenic dendritic cells. In lymph nodes, a greater percentage of residual MHC I⁺ dendritic cells (median 7.9%; range: 5-15%) was observed. For macrophages, as with dendritic cells, replacement of MHC I⁺ cells was more complete in spleens than in lymph nodes. Medians of 4.7% (range 2.9-6.4%) and 23.4% (range 15.2-28.9%) of splenic and lymph node macrophages were MHC I⁺. Surprisingly, the extent of residual host-derived macrophages was greater than for dendritic cells. There were few residual host-derived splenic (median 1.6%; range 0.8-2.1%) and lymph node (median 2.7%; range 1.4-4.8%) B cells. These data indicate that complete depletion of MHC I⁺ APCs is not required for substantial clinical protection from GVHD, and in addition support the hypothesis that the few cases of breakthrough GVHD were likely due to variable APC replacement. From these data, the important APC cell type(s) cannot be inferred, although DC seems a reasonable candidate (Banchereau et al., 1998, Nature 392:245252).

The failure of the β₂M^{-/-}→B6 CD8 recipients to develop GVHD was not due to rejection of donor CD8 cells or to the failure of donor C3H.SW marrow to engraft. C3H.SW and B6 mice express different alleles of the CD5 pan T cell surface antigen (C3H.SW express CD5.1; B6 express CD5.2). Using a monoclonal antibody against the CD5.1 allele, donor CD8 C3H.SW T cells were observed in β₂M^{-/-}→B6 chimeras. Similarly, nearly all of the CD11b and CD11c expressing cells in the β₂M^{-/-}→B6 chimeras that underwent the second transplant were MHC I⁺, demonstrating donor C3H.SW APC engraftment.

These experiments establish that in an MHC matched, multiple minor histocompatibility antigen mismatched alloBMT model analogous to most human alloBMTs, functional host APCs are absolutely required to initiate CD8⁺ T cell dependent GVHD. Also of note, “semi-professional” antigen presentation by nonhematopoietic cells was also inadequate to induce GVHD. Although there is clear evidence of cross priming in a variety of experimental situations (Matzinger et al., 1977, Cell. Immunol. 33:92-100; Bevan, 1976, J. Exp. Med. 143:1283-1288; Bevan, 1995, J. Exp. Med. 182:639-641; Carbone et al., 1989, Cold Spring Harbor Symp. Quant. Biol. 1:551-555; Huang et al., 1994, Science 264:961-965; Huang et al., 1996, Immunity 4:349-355; Srivastava et al., 1994, Immunogenetics 39:93-98; Arnold et al., 1995, J. Exp. Med. 182:885-889; Kurts et al., 1998, J. Exp. Med. 188:409-414; Carbone et al., 1990, J. Exp. Med. 171:377-387), in the β₂M^{-/-}→B6 chimeras described here, cross priming of donor derived APCs with host peptides was insufficient to generate a GVHD reaction. Radiation and chemotherapy lead to large scale cell death and release of intracellular contents, including heat shock protein/peptide completes which have been hypothesized to mediate cross priming (Udono et al., 1993, J. Exp. Med. 178:1391-1396; Lammert et at, 1997, Eur. J. Immunol. 27:923-927; Arnold et al., 1997, J. Exp. Med. 186:461-466). Although these materials would be equally available to donor or host APCs, released host antigens presented on donor APC did not stimulate GVHD.

The results presented herein could have a substantial impact on how acute GVHD is both prevented and treated. Specific targeting of host APCs prior to the conditioning regimen will prevent GVHD from occurring at all, eliminating the need for prolonged immunosuppression. The analysis of a cohort β₂M^{-/-}→B6 chimeras prior to the second GVHD inducing transplant suggests that 100% ablation of host APCs will not be necessary in order to decrease donor T cell activation and the resultant GVHD. The model of CD8-dependent, miHA specific GVHD closely mirrors the human situation, and will be useful in preclinical studies of this strategy. If successful, such an approach could both expand the range of diseases routinely treated with alloBMT to include prevalent inherited disorders such as sickle cell anemia and the thalassemias, and allow more routine use of matched unrelated and antigen mismatched hematopoietic progenitor allografts. Also, as the peripheral T cell compartment in adult hosts post alloBMT is derived from the donor graft (Mackall et al., 1997, Blood 89:3700-3707; Mackall et al., 1997, Immunol. Today 18:245-251; Mackall et al., 1993, Blood 82:2585-2594), the ability to deliver larger T cell doses without GVHD should result in more complete immune reconstitution.

These data also provide new hypotheses to explain several intriguing clinical observations in clinical allogeneic bone marrow transplantation. It has long been recognized that a subset of alloBMT recipients have self-limited GVHD (Chao et al., 1996, In: Graft-Vs-Host Disease, Ferrara et al., eds., Marcel Dekker Inc., NY). Although the remission of GVHD has been presumed to reflect a state of acquired T cell tolerance, the present data suggest that replacement of host APCs, both by passive turnover and direct elimination of host APCs by a Graft-versus-APC reaction, may be another mechanism by which GVHD is down-regulated. More recently, infusions of T cells from the original BM donors have been given to relapsed leukemia patients months to years following the initial alloBMT. Considering the high doses of T cells given, these patients have demonstrated dramatically reduced GVHD relative to what has been observed when T cells are given at the time of transplantation (Sullivan et al., 1989, New Engl. J. Med. 320:828-834; Sullivan et al., 1986, Blood 67:1172-1175; Kolb et al., 1995, Blood 86:2041-2050; Collins et al., 1994, Blood 84:333a). Other investigators have speculated that the tissue damage and high cytokine levels induced by the conditioning regimen provides a milieu that enhances the development of GVHD (Ferrara et al., 1994, Bone Marrow transplantation 14:183-184; Ferrara et al., 1993, transplantation Proceedings 25:1216-1217; Ferrara, 1993, Curr. Opinion in Immunol. 5:794-799), which would not be the case during the T cell infusion. While a lack of tissue damage and cytokine release may in part explain reduced GVHD, it is hypothesized that the host APCs that drive GVHD reactions would be replaced by donor APCs months to years after the initial BMT, thus reducing the chance that a donor CD8⁺ T cell would interact with a GVHD inducing host APC.

The experiments presented herein provide the impetus for a different strategy for reducing GVHD, namely, host APC depletion. This strategy is therefore free of the problems associated with T cell depletion of marrow allografts, such as failure of engraftment, poor immune reconstitution, and lack of immunoreactivity against the tumor. If these potential benefits could be realized clinically, the scope and efficacy of alloBMT could be dramatically expanded, resulting in more effective treatment of many leukemias and neoplasms as well as cure of genetic stem-cell based defects such as sickle cell anemia and thalassemia.

### Example 2

### Evidence for Depletion of Cells in Normal Host Animals

Having demonstrated that mice having genetically impaired antigen presenting cells were resistant to the induction of acute GVHD, experiments to demonstrate proof of principle that this could be accomplished in a non-genetic fashion in normal host animals were conducted. Such an approach models the clinical situation in humans. Thus, the feasibility of antibody mediated dendritic cell depletion was assessed in the experiments described herein. This approach has been used to deplete lymphocyte subsets in mice and has been approved for treatment of human malignancies (Baselga et al., 1998, Cancer Res. 58(13):2825-2831; Bolognesi et al., 1998, Brit. J. Haematol. 101(1):179-188; Collinson et al., 1994, Internatl. J. Immunopharmacol. 16(1):37-49; Conry et al., 1995, J. Immunotherapy with emphasis on Tumor Immunology 18(4):231-241; Francisco et al., 1998, Leukemia and Lymphoma 30(3-4):237-245; Ghetie et al., 1997, Mol. Med. 3(7):420-427; Reitman et al., 1998, Adv. Drug Deliv. Rev. 31(1-2):53-88; Maurer-Gebhard et al., 1998, Cancer Res. 58(12):2661-2666).

The integrin, CD11c, was selected as a target antigen. CD11c is used to identify murine dendritic cells (Maraskovsky et al., 1996, J. Exp. Med. 184(5): 19531962). It has been shown to be expressed on all subsets of dendritic cells. Hamster anti-CD11c hybridoma 33D1 was purchased from the American Tissue Culture Cell repository (Metlay et al., 1990, J. Exp. Med. 171(5):1753-1771). Antibody was generated by both tissue culture growth and ascites production. C57BL6/J mice received intraperitoneal (i.p.) injections with 500 μg of 33D1 or an equal volume of phosphate buffered saline (PBS) on two consecutive days. Mice were sacrificed 3-5 days after injection to assess the impact of 33D1 administration.

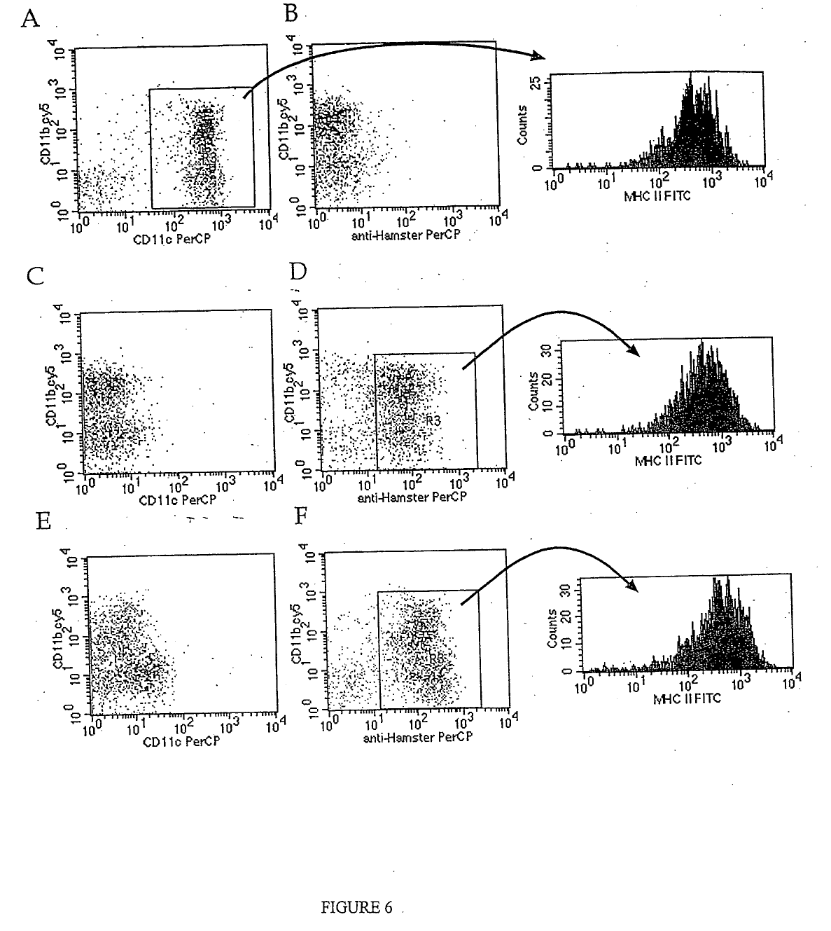

Dendritic cell enriched cell preparations obtained from spleens and lymph nodes were assessed for whether the in vivo delivered 33D1 was bound to dendritic cells. The results for the spleen cells are displayed in FIG. 6; similar results were obtained in the case of lymph node cells. Dendritic cells were identified using 4 color flow cytometry by their failure to stain with antibodies directed against myeloid (Gr-1), erythroid (TERR 119), T cell (CD3) or B cell (CD45R; B220) markers and their expression of CD11b, CD 11c, and MHC II. Dendritic cells display a classic immunophenotype of CD11c⁺ MHC II⁺ with or without expression of CD 11b. When flow cytometry was performed, a second biotin conjugated anti-CD11c antibody, clone HL3, purchased from Pharmingen (San Diego, Calif.) was used. Prior staining with 33D1 prevents binding of HL3 to the cells.

Dendritic cells obtained from PBS treated mice were readily detected using HL3 (FIG. 6, Panel A). Preincubation of the cells with 33D1 prevented detection with HL3 (FIG. 6, Panel B). The use of biotin labeled monoclonal antibodies directed against hamster IgG restored the ability to identify CD11c expressing cells (FIG. 6, Panel F). In spleen and lymph node cells obtained from in vivo 33D1 treated mice, staining with HL3 was reduced nearly 100 fold (FIG. 6, Panel E), an effect equivalent to ex-vivo blockade as shown in FIG. 6, Panel C. Staining using an anti-hamster reagent again facilitated the identification these cells (FIG. 6, Panel F). Thus, in vivo treatment with 33D1 is capable of binding a high percentage of CD11c molecules in 100% of dendritic cells.

Although 33D1 binding did not eliminate these cells, this experiment provides proof of principle for the use of toxin conjugated or radiolabeled antibodies directed against CD11c or other antigens. In addition, it has been shown that Page: 42 saporin-conjugated immunotoxins can deplete DCs. These have targeted MHCII, CD11c, the human mannose receptor (expressed on human DCs in vitro and in mice transgenic for the human mannose receptor) and against the c-type lectin DEC205 (in vivo in mice).

### Example 3

### Function of Donor APCs in CD8-Dependent GVHD

Methods for GVL and GVHD Experiments

Mice

Mice were 7-10 weeks old. C3H.SW mice were purchased from The Jackson Laboratory. B6 and B6 Ly5.1 congenic mice were obtained from the National Cancer Institute. IA—chain-deficient mice (H2-Abl^{-/-} Ly5.1⁺) mice were obtained from Taconic. C3H.SW (H-2^{b}) B2m^{-/-} mice were made by crossing C3H.SW mice with C3H/HeJ B2m^{-/-} mice (Jackson Laboratory). The absence of MHC I and homozygosity of H-2^{b} was confirmed by flow cytometry of peripheral blood.

Cell Purifications

CD8 cells were purified from lymph nodes by negative selection, as described (Matte et al. 2004) using biotin-conjugated antibodies against CD4 (clone GK1.5; lab-conjugated), B220 (clone 6B2; lab-conjugated), CD11c (clone HL3; BD Pharmingen) and CD11b (clone M1/70; BD Pharmingen), followed by streptavidin-conjugated magnetic beads (Miltenyi Biotec) and separation on an AutoMACS (Miltenyi Biotec). CD8 cells were >90% pure with CD4 T-cell contamination of <0.2%. Bone marrow T cells were depleted with anti-Thy1.2 magnetic microbeads (Miltenyi Biotec.).

GVHD Transplant Protocol

All transplants were performed according to protocols approved by the Yale University Institutional Animal Care and Use Committee. To deplete NK cells that could reject MHC I-bone marrow, all B6 Ly5.1 mice, including those that received wild-type donor bone marrow, were injected intraperitoneally with 200 g of anti-NK1.1 (clone PK13K) (Shlomchik et al. 1999; Bix et al. 1991) on days-2 and -1 before transplantation. On day 0, mice received 1,000 cGy of irradiation followed by reconstitution with 7×10⁶ T cell-depleted C3H.SW (Ly5.2) or C3H.SW B2m−/− (Ly5.2) bone marrow, with 0 or 2-3×10⁶ wild-type C3H.SW CD8 cells.

GVHD Scoring

Mice were weighed and scored for GVHD 2-3 times a week. Weights from mice that died or were sacrificed were included in averages for subsequent time points at the last value recorded. Cutaneous GVHD was assessed in eight areas for thinning fur, alopecia or ulcerations. The minimum clinical criterion for cutaneous GVHD was fur loss in one or more areas.

Analysis of DC Engraftment

Spleens were digested with collagenase as described (Shlomchik et al. 1999). To distinguish residual recipient (Ly5.1⁺ MHC I⁺), C3H.SW B2m^{-/-} donor (Ly5.1⁻ MHC I⁻) and C3H.SW (Ly5.1⁻ MHC I⁺)-derived DCs, preparations were stained with antibodies against Ly5.1 (FITC; Pharmingen), CD11c (PE; Pharmingen), a cocktail of biotin-conjugated antibodies against Gr-1 (Pharmingen; clone RB6-8C5), CD19 (Pharmingen; clone 1D3), TER119 (Pharmingen) and Thy1.2 (clone 30H12; lab conjugated) and MHC I (cy5; clone 2B-11-5S; lab conjugated). Cells were washed and stained with streptavidin-PerCP (Pharmingen). Live DCs were identified as being negative for propidium iodide and PerCP and CD11c⁺.

Histologic Analysis

Mice were sacrificed 40 and 61 d after transplantation. Tissues were fixed in 10% phosphate-buffered formalin, paraffin-embedded, sectioned and stained with hematoxylin and eosin. Slides were read by pathologist expert in skin and gastrointestinal disease without knowledge as to experimental group. Scoring was performed as described (Shlomchik et al. 1999; Anderson et al. 2003).

Immunofluorescence Microscopy

Tissues were fixed in 0.7% formaldehyde overnight, followed by dehydration in 30% sucrose and freezing in Tissue-TeK OCT compound (Sakura Finetek). Sections (5 m each) were incubated with anti-mouse CD45.1-FITC (Pharmingen), anti-mouse CD45.2-biotin (Pharmingen) or anti-mouse MHC II-Alexa 647 (clone TIB120; lab-conjugated) overnight at 4° C. After washing, streptavidin-conjugated Alexa 568 (Molecular Probes) was added when staining for CD45.2. Slides were counterstained with DAPI. Sections were photographed with a SPOT camera (Diagnostic Instruments). Three-color pictures were reconstituted with Photoshop 7 (Adobe). FITC and Alexa 568 were rendered in green, whereas anti-MHC II and DAPI were assigned red and purple, respectively.

Retrovirus Production

MSCV2.2 expressing the human BCR-ABL1 (p210) cDNA and a nonsignaling truncated form of the human low-affinity NGFR receptor driven by an internal ribosome entry site (Mp210/NGFR) was a gift from W. Pear (University of Pennsylvania School of Medicine). Retroviral supernatants were generated by transfection of BOSC ecotropic retrovirus-producing cells as described (Matte et al. 2004; Pear et al. 1993, 1998).

Progenitor Infections

p210-infected progenitors were generated as described (matte et al. 2004; Pear et al. 1998). Briefly, B6 mice were injected on day-6 with 5 mg of 5-fluorouracil (5FU; Pharmacia & Upjohn). On day-2, bone marrow cells were harvested and cultured in prestimulation media (DME, 15% FBS, 5% WEHI supernatant, IL-3 (6 ng/ml), IL-6 (10 ng/ml) and SCF (10 ng/ml). All cytokines were from Peprotech. On days-1 and 0 cells underwent ‘spin infection’ with p210-expressing retrovirus.

GVL Transplant Protocol

On day 0, B6 hosts received two 450-cGy fractions and were reconstituted with 5×10⁶ T cell-depleted C3H.SW or C3H.SW B2m^{-/-} bone marrow with 7×10⁵ B6 bone marrow cells that underwent spin infection, with or without 1.2×10⁶ purified wild-type C3H.SW CD8⁺ T cells.

Statistical Methods

Significance for differences in weights was calculated by an unpaired t-test. P values for incidence of skin disease were calculated by 2 (if one group had no events) or by log rank Mantel-Cox if events occurred in both groups. P values for histology comparisons were calculated by Mann-Whitney.

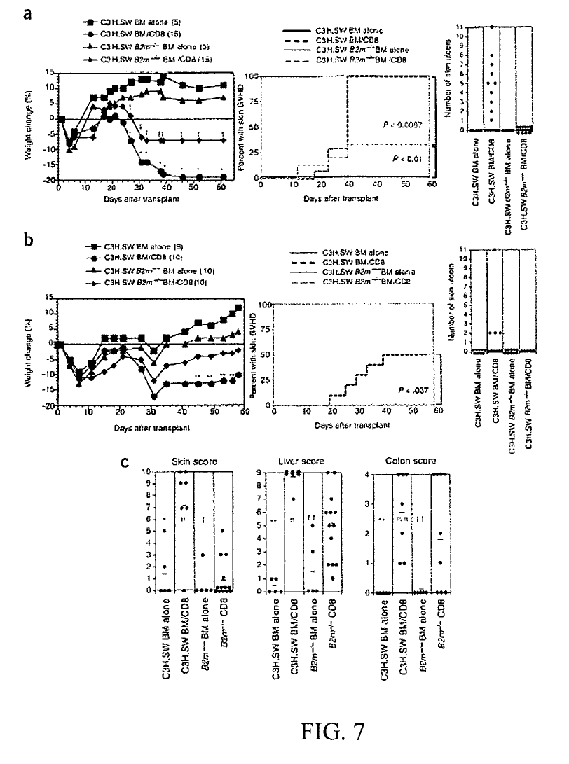

To ask whether donor APCs function in CD8-dependent GVHD, the same C3H.SW (H-2^{b}) B6 (H-2b) MHC-identical, multiple minor H antigen-mismatched mouse model was used in which it was previously established that functional recipient APCs are required for GVHD (Matte et al. 2004). To impair donor APCs, the β-2-microglobulin (B2m^{-/-}) allele were crossed from C3H/HeJ to C3H.SW. APCs that develop from B2m^{-/-} donor bone marrow are MHC I⁻ and therefore cannot prime donor CD8 cells. CD8 recipients in both the wild-type and B2m^{-/-} groups developed GVHD, manifested by hunched posture and weight loss (FIG. 7a). However, GVHD incidence and severity were greater in recipients of wild-type bone marrow and CD8 cells. In particular, skin disease was rare and mild in recipients of B2m^{-/-} bone marrow (FIG. 7a). Skin disease developed in only 5 of 15 recipients of B2m^{-/-} bone marrow and wild-type CD8 cells, compared with 15 of 15 recipients of wild-type bone marrow and CD8 cells (P<0.0007). Notably, no recipients of C3H.SW B2m^{-/-} bone marrow and CD8 cells developed skin ulcerations, compared with 10 of 15 recipients of C3H.SW bone marrow and CD8 cells, many of which had multiple ulcers (FIG. 7a). Six deaths resulting from GVHD were observed in recipients of C3H.SW bone marrow and CD8 cells, whereas no deaths were observed in recipients of B2m^{-/-} bone marrow and CD8 cells. A second experiment in which GVHD was less severe in all groups, confirmed these results (FIG. 7b).

Pathologic analysis confirmed the clinical findings (data from one of two experiments, FIG. 7c). GVHD pathology scores of liver and colon were significantly higher in recipients of B2m^{-/-} and wild-type CD8 cells than in mice that received only B2m^{-/-} bone marrow. This definitively establishes that functional donor APCs are not required for histological GVHD. Skin and liver GVHD were much more severe in recipients of C3H.SW bone marrow and CD8 cells, compared with recipients of C3H.SW B2m^{-/-} bone marrow and wild-type CD8 cells. Overall, colonic involvement was mild, but there was a trend towards being more severe in recipients of wild-type bone marrow (P<0.2). Thus, although GVHD developed in recipients of MHC I⁻ bone marrow, it was pathologically and clinically less severe.

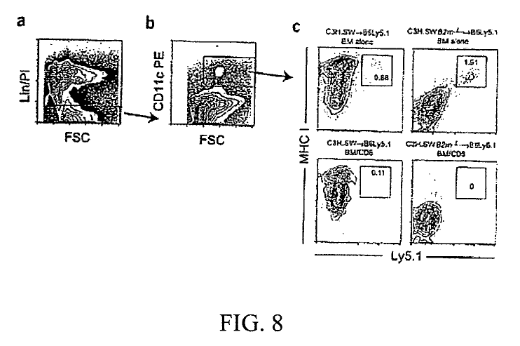

As recipients of B2m^{-/-} bone marrow still developed GVHD, an obligatory role for donor APCs in GVHD pathogenesis can be excluded. One interpretation of these data is that once primed on host APCs, sufficient CD8 expansion and maturation ensues such that further contact with professional APCs is not required. Alternatively, residual recipient APCs may survive to stimulate previously activated or naive CD8 cells. Therefore, spleens of GVHD mice were analyzed by flow cytometry for the presence of residual host dendritic cells (DCs). Residual host and donor bone marrow-derived cells were identified by expression of Ly5.1 (host) and Ly5.2 (donor, wild-type and B2m^{-/-}). Donor B2m^{-/-} cells were distinguished from cells that might have contaminated CD8 preparations by the absence of MHC I. Representative recipients of wild-type or B2m^{-/-} bone marrow with clinical GVHD (subsequently confirmed histologically) were sacrificed 40 d after transplant. Residual recipient DCs comprised <0.01% of splenic DCs in mice reconstituted with B2m^{-/-} bone marrow and wild-type CD8 cells, and were undetectable in four of six mice analyzed (FIG. 8). Similar data was collated for F4/80⁺ macrophages. Residual host DCs were also essentially absent in three of three recipients of wild-type donor bone marrow (0.11%, 0% and 0% of DCs). Donor-derived B2m^{-/-} and wild-type DCs were well-engrafted well (FIG. 8), with a higher frequency of B2m^{-/-} DCs than wild-type DCs in CD8 recipients (1.6% compared with 0.43%; n=3 and 4 per group, respectively).

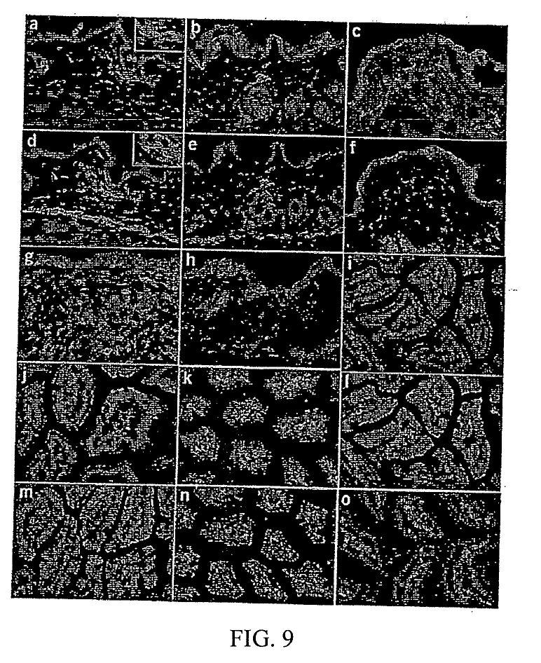

The possibility that host DCs persisted in tissues was also considered. Skin and bowel were analyzed from recipients of B2m^{-/-} bone marrow and wild-type CD8 cells by immunofluorescence microscopy for the presence of donor and host APCs (FIG. 9). Frozen sections were stained for Ly5.1 (host) or Ly5.2 (donor), and for MHC II to identify APCs. Skin (FIG. 9a) and bowel (FIG. 9i) from recipients of B2m^{-/-} bone marrow and CD8 cells with GVHD had MHC II⁺ cells, but these were Ly5.1⁻. Conversely, there was strong staining for Ly5.2 that colocalized with the MHC II staining in skin (FIG. 9d) and bowel (FIG. 9l), demonstrating donor APC engraftment. Skin from a recipient of wild-type bone marrow and CD8 cells with severe GVHD showed extensive infiltration with donor Ly5.2⁺ cells (FIG. 9g) including all MHC II⁺ cells. There was also substantial MHC II staining on bowel epithelial cells, possibly stress-induced (Bland et al. 1992), especially in mice with GVHD. This staining was specific, as no MHC II expression was observed in MHC II-mice (FIG. 9o).

Overall, these data suggest that if there were residual host APCs in recipients of B2m^{-/-} bone marrow and CD8 cells, they were largely below the level of detection and that at a minimum, most APCs in recipients of B2m^{-/-} bone marrow were donor-derived. In addition the number of residual host APCs was not increased in recipients of wild-type bone marrow, making it unlikely that increased GVHD in this group was the result of greater survival of host APCs. Taken together, these data indicate that residual recipient APCs were unlikely to have a major function in maintaining GVHD at the time of analysis.

### Example 4

### Function of Donor APCs in CD8-Mediated GVL

In addition to causing GVHD, alloreactive T cells mediate GVL. As the data suggested that targeting donor APCs may be effective in decreasing GVHD, it was also asked whether donor APCs are required for GVL. GVL against a mouse model of chronic phase chronic myelogenous leukemia (CP-CML) induced by a retrovirus that expresses the BCR-ABL1 (p210) fusion cDNA (Matte et al. 2004) was also being studied in these investigations. When lethally irradiated mice receive p210-transduced mouse bone marrow, they develop a myeloproliferative disease marked by a high peripheral white blood cell count and splenomegaly (Pear et al. 1998; Daley et al. 1990) with hematopoiesis dominated by maturing myeloid cells. A difference between mouse CP-CML and human CP-CML is that mice succumb to leukemic infiltration of the lung. The retrovirus also expresses a nonsignaling form of the low-affinity nerve growth factor receptor (NGFR), which allows detection of infected cells by flow cytometry.

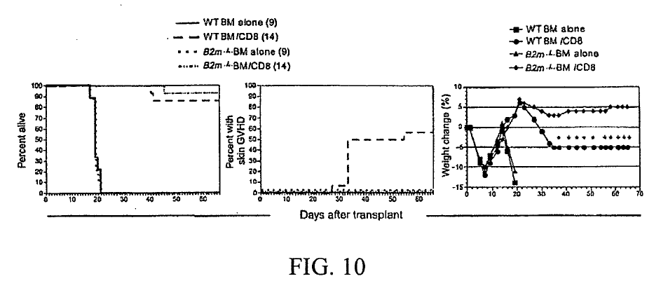

B6 mice were irradiated and reconstituted with p210-transduced B6 bone marrow, T cell-depleted bone marrow from either C3H.SW B2m^{-/-} or wild-type C3H.SW mice, with or without 1.2 106 C3H.SW CD8 cells. All mice that did not receive CD8 cells died from CP-CML 18-21 d after transplant, whereas only 1 of 14 CD8 recipients in each group died from CP-CML (FIG. 10). As in the previous GVHD experiments, recipients of B2m^{-/-} bone marrow and CD8 cells developed less weight loss and no skin disease, whereas 8 of 14 recipients of wild-type bone marrow and CD8 cells developed skin GVHD (FIG. 10). Even so, none of the surviving recipients of B2m^{-/-} bone marrow and CD8 cells had splenic leukemic cells when sacrificed 60 d after transplantation. Thus, donor-derived APCs were not required for GVL against CP-CML, but impairment of donor APCs decreased GVHD.

These data show that CD8-mediated GVHD across only minor H antigens occurs independent of donor-derived APCs. Therefore, after initial priming, alloreactive donor CD8 cells can expand and mature into effectors without participation by donor APCs. A key question is whether alloreactive CD8 cells in these mice become independent of hematopoietic APCs, or whether residual recipient APCs continue to prime donor CD8 cells. That few, if any, residual host APCs were found in recipients of B2m^{-/-} bone marrow and wild-type CD8 cells further suggests that donor CD8 cells can indeed become independent of hematopoietic APCs altogether. These data extend the results of prior studies that used transgenic T cells and in vitro assays to study the required duration of APC-CD8 T-cell contact (Kaech et al. 2001; van Stipdonk et al. 2001) to an entirely in vivo system with polyclonal T cells. Because mice were not sacrificed at earlier time points to assess APC turnover, it is not known when donor CD8 cells became independent of further contact with host APCs. Nevertheless, the more general point is that at some time after GVHD initiation, alloreactive T cells can become APC-independent.

However, donor APCs do have an important role in GVHD pathogenesis, as GVHD was much less severe in recipients of B2m^{-/-} bone marrow. It is most probable that donor APCs cross-presenting host antigens promote GVHD by maintaining or expanding the initial pool of alloreactive CD8 cells generated by initial priming on host APCs. Although this initial population of alloreactive T cells is adequate for a limited form of GVHD, it is insufficient for maximal disease. Without cross-priming by donor APCs, these T cells are effectively deprived of antigen. Donor APCs could prime donor CD8 cells in secondary lymphoid organs, in GVHD target tissues or in both. Activity in target tissues may be particularly important for skin GVHD, which was nearly absent in recipients of B2m^{-/-} bone marrow. In addition to priming infiltrating alloreactive cells, tissue DCs may also produce chemokines that promote further CD8⁺ T-cell infiltration. Although the absence of MHC I⁺ donor APCs capable of cross-presenting host antigens is the simplest and most likely explanation for reduced GVHD in recipients of C3H.SW B2m^{-/-} bone marrow, it cannot be formally excluded that some other unknown property of B2m^{-/-} cells reduces GVHD.

The augmentation of ongoing GVHD by donor APCs is in contrast to the inability of engrafting or long-term resident donor APCs to initiate GVHD in the same model. Several differences between the situation early after allogenic stem cell transplantation (alloSCT) and after GVHD has been established might explain this disparity. Early after alloSCT, alloreactive CD8⁺ T cells are rare, and donor APCs that cross-present sufficient host peptide may be too infrequent for efficient GVHD initiation. In contrast, host APCs are effective because they all endogenously present high levels of host peptides. However, the increased frequency of alloreactive T cells after priming on host APCs may make it more probable that they will subsequently encounter a donor APC cross-presenting sufficient host peptides (Mintern et al. 2002). Antigen-experienced T cells also have reduced activation requirements and thus donor APCs that cross-present low levels of host peptides may be efficacious (Lezzi et al. 1998; Croft et al. 1994; Kedi et al. 1998; Zimmermann et al. 1999; Cho et al. 1999; London et al. 2000; Sprent et al. 2002). Finally, features of the GVHD environment, including proinflammatory cytokines and increased availability of TLR ligands, may promote cross-presentation (Le Bon et al. 2003; Singh-Jasuja et al. 2000; Hu et al. 2002; Hoffmann et al. 2001; Kopp et al. 2003). Regardless of explanation, donor APCs clearly function in CD8-mediated GVHD and these experiments identify them as a new target for GVHD treatment.

Because most alloSCTs are performed for treatment of malignant diseases, it was important to ask whether donor APCs are absolutely required for GVL. In this model, donor APCs are dispensable for GVL. Both mouse CP-CML and the transplant protocol are clinically relevant. First, mouse CP-CML is a primary neoplasm, caused by the identical genetic abnormality as human CP-CML, which recapitulates the characteristic myeloproliferative syndrome. This distinguishes it from most model leukemias used in GVL studies, which are cell lines that share neither phenotype nor genetic etiology with common human leukemias. Second, the transplant model is MHC-identical and minor H antigen-disparate, as are most human alloSCTs. Finally, in clinical transplantation, GVL is uniquely effective against CP-CML (Horowitz et al. 1990; Kolb et al. 1995). These data do not exclude a role for donor APCs in GVL against other neoplasms; however, in some clinical settings the initial GVL generated on host APCs may be sufficient to eliminate clonogenic leukemia cells. Human CP-CML cells can differentiate into DCs in culture (Choudhury et al. 1997), and thus it is possible that mouse CP-CML cells do the same. These cells might directly prime donor T cells to alloantigens.

The work presented here now provides a complete picture of the roles of both donor and host APCs in a single CD8-dependent model of acute GVHD. The findings give further support to strategies that target APCs, both host and donor, as a means of preventing GVHD. Moreover, these data provide a rationale for targeting donor APCs in the treatment of established GVHD. The result that donor APC impairment did not affect GVL suggests that eliminating donor APCs to treat ongoing GVHD may not compromise GVL against CP-CML. In engrafted recipients, donor-derived APCs initiate adaptive immune responses to pathogens, and their elimination may increase the susceptibility to infection, as do all current immunosuppressive strategies, including those that target T cells. In vivo T cell-depleting reagents, such as anti-thymocyte globulin or Campath-1H, used to treat refractory GVHD cause a long-lasting if not permanent reduction in T-cell number and antigen receptor diversity (Chakrabarti et al. 2002; Cragg et al. 2000). In contrast, DCs can fully reconstitute from hematopoietic progenitors, and thus, APC depletion might be efficacious without indefinitely increasing the susceptibility to infections.

### Example 6

### Requirement of CD80/86 Expression on Either Donor or Host APCs for Cutaneous cGVHD

Methods

Mice

BALB/c mice were purchased from the National Cancer Institute (Frederick, Md.). B10.D2.oSN, CD40^{-/-} (on a BALB/c background) and B6.C mice (C57Bl/6 mice onto which the H-2^{d} MHC locus has been backcrossed) were purchased from the Jackson Laboratory (Bar Harbor, Me.). CD80/86^{-/-} mice (on a BALB/c background) were backcrossed for more than 10 generations from the original knock-out mice (Borriello et al. 1997) kindly provided by Arlene Sharpe (Brigham and Women's Hospital, and Harvard Medical School, Boston, Mass.). B6.C, CD80/86^{-/-} (B6.C), and CD40^{-/-} (B6.C) were bred and housed under specific pathogen-free conditions at Yale University School of Medicine. All recipients were 8 to 12 weeks at the time of initial transplantation.

Bone Marrow Transplantation (BMT)

Donor animals (B10.D2.oSN, B6.C, CD80/86^{-/-} [B6.C], CD40^{-/-} (B6.C]) and recipient animals (BALB/c, CD80/86^{-/-} [BALB/c], CD40^{-/-} [BALB/c]) were all H-2 d. Recipient mice received total body irradiation (TBI) from a ¹³⁷Cs source as either a single dose of 850 cGy or 2 doses of 425 cGy separated by 3 hours. Three to 5 hours following the last irradiation dose all recipients received 0.8×10⁷T-cell-depleted bone marrow (BM) suspended in injection buffer (I×phosphate-buffered saline, 10 mM HEPES [N-2-hydroxyethylpiperazine-N′-2-ethanesulfonic acid], 2.5% acid citrate dextrose anticoagulant, 0.5% penicillin-streptomycin) with or without WT B10.D2 or B6.C donor T cells via tail-vein injection. Total spleen cell dose was 10⁷ cells/recipient; the purified CD4 cell dose was 2×10 6. Animals were given water supplemented with trimethoprim-sulfamethoxazole for 2 weeks following BMT.

Chimeric Recipients

(Donor host) and (host→host) chimeric recipients were prepared by transplanting B10.D2 or BALB/c BM, respectively, into BALB/c mice, as described in “Bone marrow transplantation (BMT).” Recipients were rested for more than 2 months to allow full reconstitution of the hematopoietic system by donor cells. Lymph nodes (LNs) and spleens of (donor host) chimeras contained less than 2% recipient-type dendritic cells as determined by flow cytometry. Chimeric recipients were then used in a standard GVHD-inducing BMT.

Cell Separations

BM cells were isolated and prepared as previously described (Anderson et al. 2004; 2003). Remaining Thy1.2-positive cells were routinely less than 0.5% of BM cells as determined by flow cytometry.

Splenic CD4 cells were isolated using BioMag beads (QIAGEN, Valencia, Calif.) as previously described (Anderson et al. 2003). For experiments using CD80/86^{-/-} recipients and pure CD4 cells, CD4 cells were further enriched after BioMag-based purification as follows: BioMag-enriched CD4 cells (70%-80% pure) were incubated with biotinylated anti-CD4 (GK1.5) for 30 minutes on ice. Cells were washed once in magnetic cell sorting (MACS) buffer and then incubated with streptavidin-conjugated microbeads (Miltenyi Biotech, Auburn, Calif.) for 30 minutes at 4° C. CD4 cells were positively selected using an AutoMACS (Miltenyi Biotech), and resulting cells were more than 98% CD4 as determined by flow cytometry.

Clinical and Pathologic Scoring

Animals were analyzed for clinical and pathologic cGVHD as previously described (Anderson et al. 2003). The following scoring system was used: healthy appearance=0; skin lesions with alopecia less than 1 cm 2i n area=1; skin lesions with alopecia 1 to 2 cm 2 in area=2; skin lesions with alopecia more than 2 cm 2 in area=3. Additionally, animals were assigned 0.3 point each for skin disease (lesions or scaling) on ears, tail, and paws. Minimum score=0, maximum score=3.9. Incidence and clinical score curves represent all mice with scores 0.6 or higher. Final scores for dead animals were kept in the data set for the remaining time points of the experiment. Slides of skin were scored by a dermatopathologist on the basis of dermal fibrosis, fat loss, inflammation, epidermal interface changes, and follicular dropout (0-2 for each category). Minimum score was 0, and maximum score was 10. Colon slides were scored by a gastrointestinal pathologist (D. J.; blinded to experimental groups) on the basis of inflammation and apoptosis (0-3 for each category). Minimum score was 0, and maximum score was 6.

Statistical Methods

The significance of differences in cGVHD incidence was calculated by log-rank Mantel-Cox. The significance of differences between clinical scores and pathology scores were calculated by the Mann-Whitney nonparametric test. Significance of differences of weight changes was calculated by Student t test.

The overall goal was to determine the relative contributions of donor- and host-derived APCs in the genesis of cGVHD. Prior studies in this model determined that cGVHD is initiated by naive donor CD4 cells (Anderson et al. 2003). Because the signals delivered by CD28: CD80/86 interactions are known to be critically important for activation of naive CD4 cells, CD80/86^{-/-} were chosen as donors and/or recipients in the cGVHD experiments. This was the optimal choice for inactivating both donor and host APCs. MHC class II-deficient donors or recipients could not be used because the H-2^{d} haplotype contains 2 MHC class II chain genes, and double knockouts are not available. Similarly, invariant chain knockouts and class II transactivator knockouts, in which MHC class II expression has been reported to be reduced, are not suitable because they have substantial MHC class II expression on dendritic cells, especially under inflammatory conditions (Kenty and Bikoff, 1999).

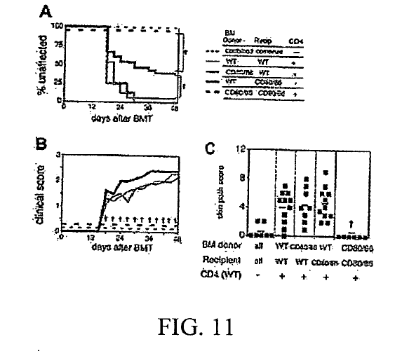

To test the validity of this approach, it was first determined whether cGVHD required CD28:CD80/86 interactions. CD28:CD80/86 signaling was eliminated by all APCs (donor and host) by transplanting CD80/86^{-/-} BM and highly purified wild-type (WT) CD4 cells into CD80/86^{-/-} recipients. Strikingly, no clinical cGVHD developed in these mice, in contrast to WT recipients of WT BM and CD4 cells (FIG. 11A). Therefore, donor CD4 cells absolutely require signals from CD80/86 to mediate clinical cGVHD of the skin in this model, validating the use of CD80/86 knockouts to identify the roles of donor and host APCs individually.

In the next set of experiments, cGVHD in CD80/86^{-/-} BM+CD4 T cells WT were compared versus WT BM+CD4 T cells CD80/86^{-/-} to debilitate antigen presentation by donor or host APCs, respectively. Cutaneous cGVHD developed in both groups, demonstrating that donor or recipient APCs are sufficient to initiate disease (FIG. 11A). However, the incidence of cutaneous cGVHD in CD80/86^{-/-} recipients was less than that in WT recipients (FIG. 11A). This suggested that recipient APCs are more important for eliciting cutaneous cGVHD than donor APCs. In support of this, cGVHD incidence was not reduced when CD80/86^{-/-} BM+CD4 T cells were given to WT recipients, suggesting that, when host APCs are intact, reconstitution with defective donor APCs does not affect disease.

Although the incidence of cGVHD was reduced in WT CD80/86^{-/-} mice, the extent of disease among affected mice as measured by clinical score was indistinguishable from WT WT or CD80/86^{-/-} WT cGVHD mice (FIG. 11B). Consistent with the clinical score, histologic disease was similar in all affected mice (FIG. 11C). Thus, regardless of which APCs were impaired, once cGVHD developed, it was similar to that seen in WT WT mice.

### Example 7

### Host-Type APCs not Required to Initiate cGVHD

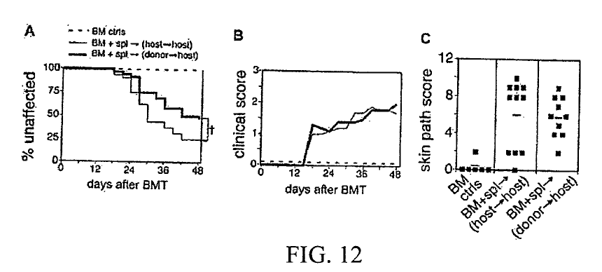

To address whether host-type APCs needed to be resident at the time of transplantation for optimal GVHD induction, cGVHD in (donor→host) and control (host→host) chimeras were compared. cGVHD developed in (donor host) chimeras (FIG. 12), even though more than 98% of APCs were donor-type (flow cytometry, data not shown). The onset and incidence of cGVHD in (donor host) chimeras was reduced to a slight but statistically significant (P<0.01) degree compared with (host→host) chimeras (FIG. 12A). Severity and pathology scores were indistinguishable in the 2 groups (FIG. 12B-C). Thus, cGVHD can be initiated by donor APCs, but host APCs are required for the maximal penetrance of skin disease, consistent with the data using CD80/86^{-/-} recipients. It was reported in another model that Langerhans cells in skin remained host type unless donor T cells were also transferred. In a fully allogeneic model, persistence of host Langerhans cells correlated with severity of GVHD (Merad et al. 2004). Although Langerhans cells in the recipients may have remained host-type, GVHD was actually reduced in such mice, indicating a role for host-type APCs other than Langerhans cells.

### Example 8

### CD40 Important but not Required on Donor APCs when Host APCs are Inactivated

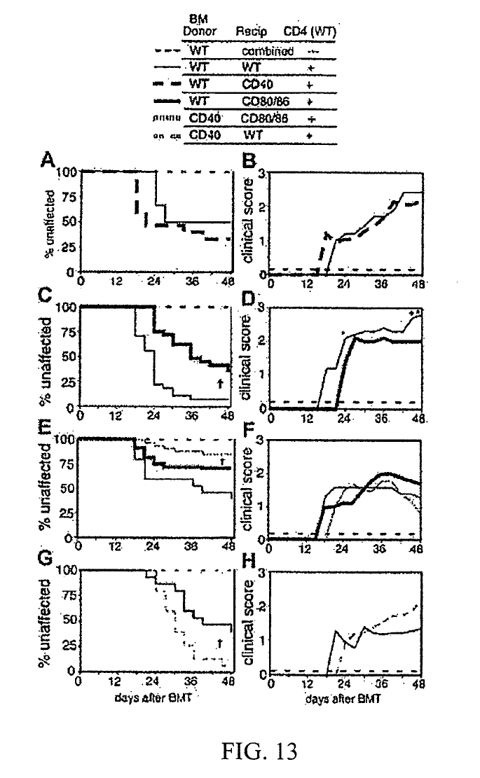

As noted in “CD80/86 costimulation in cGVHD is independent of CD40,” when donor APCs are intact, CD40 expression on the host also had no effect (FIG. 13A-B). Because both host and donor APCs can function to promote cGVHD, it was important to determine whether there was a role for CD40 when only donor APCs can activate alloreactive T cells. Therefore, CD80/86^{-/-} recipients were infused with donor BM that lacked CD40 expression, along with WT donor CD4 T cells. Cutaneous GVHD was reduced but not eliminated in CD80/86^{-/-} recipients of CD40^{-/-} BM compared with recipients of WT BM (FIG. 13E-F). This contrasts with the situation when host APCs are intact, as CD40 expression on the donor BM had no detectable role in promoting skin disease in WT recipients (FIG. 13G-H). In fact, if anything, when host APCs are intact the absence of CD40 on donor BM leads to increased skin disease incidence (although not increased severity; FIG. 13H). This may be because when donor APCs lack CD40 they do not engage counter-regulatory mechanisms such as up-regulation of CD80/86 that can in turn ligate cytotoxic T lymphocyte-associated antigen 4 (CTLA-4) and may also be important for the function of regulatory T cells (Salomon et al. 2000). Similar paradoxical effects have been seen in autoimmunity and transplantation (Salomon et al. 2001). Thus, CD40 has a unique, but not absolutely required, function in promoting GVHD when APCs from the donor are the exclusive means of activating alloreactive CD4 T cells. It is therefore possible that donor APCs taking up exogenous antigens may differ from resident host APCs in their requirements for activation via CD40. In toto, these results may have implications for the mechanism of CD40L-based inhibition of GVHD (Blazar et al. 1997).

### Example 9

### Dependence of Gut cGVHD on Intact Donor APCs

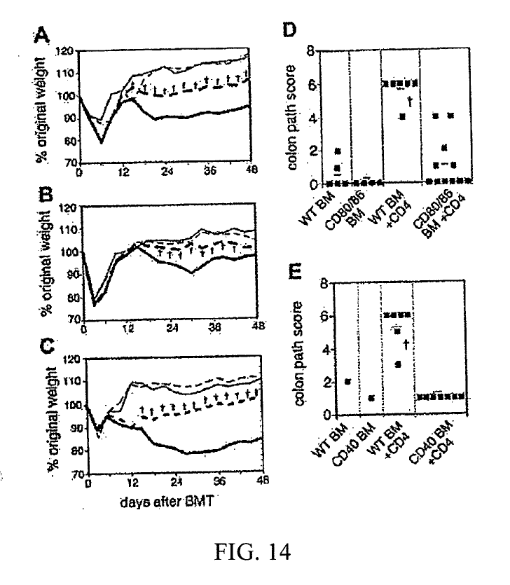

The previously described experiments focused on the prominent target organ, skin. However, the unexpected and consistent findings of increased weight loss (FIGS. 14A-B) and diarrhea were noted in recipients of WT BM as compared with non-WT BM. Following recovery from irradiation, CD4 recipients of CD80/86^{-/-} BM regained and maintained their original weight, while CD4 recipients of WT BM never returned to their pretransplantation weight (FIG. 14A). Mice that received WT or CD80/86^{-/-} BM alone without CD4 cells had equivalent weights at day 18 and later. Similarly, mice that received CD40^{-/-} BM and spleen cells had significantly higher average weight than recipients of WT BM and spleen cells (P<0.01) (FIG. 14B). In fact, following recovery from irradiation, there was no statistical difference (P>0.05) in average weights for CD40^{-/-} BM alone controls as compared with those receiving CD40^{-/-} BM and spleen cells, with the single exception of day 33. In contrast, recipients of WT BM and spleen cells had significantly lower (P<0.01) weights than WT BM alone controls for all time points after day 21 (statistics not indicated on graph). As before, weights of WT BM alone versus CD40^{-/-} BM alone recipients were not statistically different. The requirement for CD40 on donor BM to mediate gut disease is similarly present when host APCs are inactivated by the CD80/86 double knockout (FIG. 14C). These data additionally show that host APCs need not express CD80/86 for gut GVHD to ensue.

The requirement for intact donor APCs in promoting gut pathology was confirmed histologically in mice that received CD80/86-deficient BM (FIG. 14D). Recipients of CD4 T cells along with WT BM had substantially higher gut pathology scores than equivalent mice that received CD80/86-deficient BM (P<0.01). Indeed, although a few of the recipients of CD80/86-deficient BM had detectable gut pathology, in aggregate their scores were statistically indistinguishable from recipients of either type of BM without donor CD4 T cells (P=0.19 and 0.73); in other words, without CD80/86 expression on donor BM, there was no statistical evidence that donor T cells caused GVHD compared with BM-alone controls. Gut pathology was examined in the experiment shown in FIG. 14C, in which CD80/86^{-/-} recipients received CD40^{-/-} BM. Again, colon pathology was only observed when CD40 was intact on donor BM (FIG. 14E), corroborating the weight loss data.

These studies demonstrate that gut GVHD, as indicated by both weight loss and histopathologic disease in this model, is markedly attenuated in recipients of BM deficient in key T-cell-stimulating molecules. This suggests that donor T cells are stimulated to cause gut disease by APCs originating from the donor BM. Host APCs are not necessary as CD80/86^{-/-} recipients that received WT BM and CD4 T cells do get gut GVHD that is comparable to that induced in WT WT transplantations (data not shown). The finding that donor-derived APCs have a nonredundant function for this form of cGVHD, but not skin cGVHD, points to distinct disease-initiating requirements for different target organs of cGVHD.

Discussion

To understand the initiation of GVHD at a basic level, it is important to determine whether donor, host, or both types of APCs are necessary and sufficient to cause GVHD. It was previously shown that in a CD8-mediated miHA-incompatible model of aGVHD, host APCs were necessary for GVHD initiation (Shlomchik et al. 1999), identifying these as a target for GVHD prevention. In humans, there is ample evidence that both CD4 and CD8 T cells can mediate GVHD. While several reports have investigated and shown a role for host APCs (Shlomchik et al. 1999, Ruggeri et al. 2002; Merad et al. 2004; Korngold et al. 1983; Teshima et al. 2002; Duffner et al. 2004), there have been few reports of a role for donor APCs.

Here, this question was directly addressed by using a CD4-dependent, MHC-matched model of GVHD. Important roles for donor APCs were found in promoting the skin manifestations of cGVHD, such as fibrosis and dropout of adnexal structures. Intact host APCs were also sufficient to induce cGVHD but dispensable as long as donor APCs were competent. However, when host APCs alone were impaired, the penetrance of cutaneous cGVHD was reproducibly reduced, indicating a partially exclusive role for host APCs. The induction of cGVHD in hosts lacking CD80/86 also indicates that expression of these molecules on any host tissue is not required for GVHD and thus allows the discussion to be restricted to the effects of CD80/86 on APC function.

These results raise the question of why APC requirements differ in the CD4-dependent model of cGVHD that were used and the CD8-mediated aGVHD model previously reported (Shlomchik et al. 1999). One simple explanation is that the MHC II antigen presentation pathway incorporates exogenous antigens by design, thus facilitating presentation of host-derived miHAs by donor-derived APCs. While presentation of exogenously acquired antigen can also occur on MHC I (cross-presentation) (Carbone et al., 1990), it is less efficient and operationally is insufficient to initiate GVHD when CD8 cells alone are given in a miHA-mismatched model (Shlomchik et al. 1999). MC II-mediated presentation of host-derived miHAs by donor-derived APCs can even enable GVHD to occur when the host hematopoietic system has been replaced by the donor-type bone marrow (FIG. 12). In this case, only donor-type APCs exist, and they must present host antigens from nonhematopoietic tissues; similar evidence for the importance of miHA expressed on nonhematopoietic tissue has been obtained by Korngold and colleagues (Jones et al, 2003). In contrast, analogous chimeras in the CD8-mediated system that was studied did not get GVHD (Shlomchik et al. 1999). Aside from differences in presentation pathways, CD4 T cells may differ from CD8 T cells in their trafficking, activation requirements, and survival requirements. However, at present there is no information on which if any of these might affect differential APC requirements.

In addition to demonstrating the role of donor APCs, it is shown that the function of both donor and host APCs requires CD80/86. Thus, at some stage, for GVHD to ensue, CD4 T cells must receive CD80/86-mediated signals, presumably transduced through CD28 expressed on the CD4 T cells themselves. Costimulation by CD80/CD86 is particularly important in the activation of naive CD4 T cells (Green et al. 1994; Sperling et al. 1996; Schweitzer et al. 1998). The dependence on CD80/CD86 that was demonstrated is consistent with the recent finding that GVHD in this model is mediated only by naive T cells (Anderson et al. 2003), a result which has been extended to several different murine systems (unpublished data, B. A., January 2004, and Chen et al, 2004). Furthermore, when resident host APCs were CD80/86 deficient, GVHD incidence was reduced even though donor APCs were wild type, again arguing that CD80/86 is probably required for initial priming. However, it should be emphasized that the results do not mean that CD80/86 is a critical T-cell activator throughout the GVHD course. For example, it is plausible that initial priming could occur in the host in a CD80/86-dependent fashion, but subsequent T-cell activation required for frank GVHD could occur on donor APCs without CD80/86 function. Nonetheless, the critical role of CD80/86 at some point in the process is clearly established by the complete absence of GVHD when both donor and host are deficient.

The important role of costimulation in various models of GVHD has been studied by a number of others, mainly in MHC-disparate models. APC and costimulatory requirements, which can depend on antigen dose (Green et al. 1994; Lumsden et al. 2003), may differ from the miHC-mismatched situation that were studied. Nonetheless, in these studies, GVHD has been reduced by using CTLA4 immunoglobulin, anti-CD80/86 antibodies, or CD28-deficient T cells (Speiser et al. 1997; Yu et al. 1998; Via et al. 1996; Blazar et al. 1994, 1996). Because these prior studies used inhibitor or CD28-deficient T cells, they could not distinguish the differential roles of donor and host APCs, as done in the present work (Blazar et al. 1994) were the first to show a role for CD80/86 in a miHA-incompatible model. They used spleen cells to elicit GVHD in a setting in which CD8 cells cause GVHD that can be augmented by CD4 cells, although the latter do not cause GVHD by themselves. CTLA4-immunoglobulin delayed GVHD induced by unfractionated splenocytes, although all mice eventually succumbed to GVHD. CTLA4-immunoglobulin had no effect when GVHD was induced by CD8 cells alone. Thus, one can infer a role for CD80/86 costimulation, albeit a modest one, in priming CD4 cells that “help” CD8 responses. Again, since inhibitors were used, the roles of donor and host APCs were not distinguished. The use of knock-out mice in the present studies does allow such distinction; moreover, the current results demonstrate a primary role for CD28:CD80/86 stimulation when CD4 cells alone are directly pathogenic, rather than functioning solely as helpers of CD8-mediated GVHD.

During normal immune responses to pathogens, both CD80 and CD86 are up-regulated upon APC maturation, and this plays an important role in their function to activate naive CD4 T cells (Inaba et al. 1995). Whether up-regulation (as opposed to expression) is required in GVHD is not known. Nonetheless, one might expect that maturation of DCs, with its attendant up-regulation of CD80/86, would be important for GVHD induction. Signals through CD40 on the DCs, delivered by CD154 on CD4 T cells, can play an important role in DC maturation (Caux et al. 1994) as well as enable DCs to more optimally stimulate CD8 T cells (Ridge et al. 1998). This might be particularly important in CD4-mediated GVHD. We, therefore, studied whether the requirement for CD80/86 was downstream of CD40 signals. However, skin-targeted GVHD progressed normally even in the absence of CD40 on either donor or host APCs (FIG. 13). Thus, for skin GVHD, CD40 signaling is not obligatorily upstream of increased CD80/86 expression or other aspects of DC maturation. Presumably other means of causing DC maturation are operative, including inflammatory and Toll-like receptor signals that could be present because of tissue damage or breach of the gut barrier (Antin et al. 1992; Ferrara et al. 1993).

In studying the roles of CD80/86 and CD40 in APC function, it was a surprise to find that CD40 and CD80/86 both had non-redundant functions on donor APCs when it came to inducing gut GVHD. This finding illustrates a surprising principle that APC requirements can differ depending on the site or type of disease. In this case, donor APC function (as indicated by ability to express CD80/86) was required to mediate disease in the gut, in contrast to skin disease, even in the presence of wild-type host APCs. Without it, disease was markedly reduced, as measured by weight gain and pathologic assessment. Moreover, in contrast to the case with skin disease for which CD40 expression on either donor or host APCs was dispensable, CD40 played an important role in mediating APC activation necessary for gut GVHD. Thus, CD40 signaling is required in this setting for optimal activation of donor T cells to cause disease in the gut, albeit that a small amount of residual disease was seen in the absence of either CD40 or CD80/86 on donor cells. Only in the case in which host APCs were inactivated did a partial role emerge for CD40 on donor APCs in mediating skin disease.

It is not yet known if APC requirements differ depending on the target tissue even within the same mouse. There could be differences in the rate of APC engraftment in different tissues, leading to differential donor APC residence; for example, Langerhans cells in the skin are reported to remain largely recipient type after syngeneic transplantation while LN DCs are mainly donor type (Merad et al. 2004). Activation of gut-homing T cells in secondary lymphoid tissues could be CD80/86 dependent whereas this may not be the case for T cells that traffic to other tissues. Finally, disease in the gut may be more dynamic than in the skin, requiring persistent T-cell activation for pathogenesis. Perhaps the fibrotic reaction that ensues in the skin becomes independent of further T cell activation; this would explain why skin GVHD is relatively independent of donor APC engraftment compared with the gut. Future experiments will test these possibilities with a chance to better define different local pathogenesis mechanisms.

In addition to the mechanistic implications of the findings, there are some clinically relevant conclusions. First, the data suggest that depletion of host APCs will be effective in moderating CD4-mediated GVHD, a significant extension of the prior work that showed an essential role for host APCs in CD8-mediated GVHD. Importantly, results for CD4 and CD8 T cells were obtained in minor antigen-mismatched models, suggesting their applicability to the most common type of human stem cell transplantation. Since the model studied here also has features of cGVHD, it is possible that depletion or inhibition of host APCs at the time of transplantation will also have an effect on late complications like cGVHD, although this remains to be better tested. Second, the results add a new rationale for targeting donor APCs in vivo after transplantation, either as a means of preventing GVHD or as a method for treating established GVHD, particularly that of the gut. This could be through costimulatory molecule blockade, as demonstrated using inhibitors of CD80/86 in a variety of GVHD models (Speiser et al. 1997; Yu et al. 1998; Via et al. 1996; Blazar et al. 1994, 1996). Alternatively, this could be accomplished via reagents that physically deplete APCs. Finally, if donor APCs do play a role, particularly in gut disease, then it may be effective to deplete them at later stages as a therapy for ongoing GVHD. This concept is further supported by recent finding that donor APCs are required for maximal CD8-mediated GVHD across only miHAs (Matte et al. 2004). Direct tests of these therapeutic approaches will have to await models in which APC depletion can be carried out via reagents rather than genetically.

### Example 10

### Saporin-Conjugated Immunotoxin Against MHC II Depletes Dendritic Cells

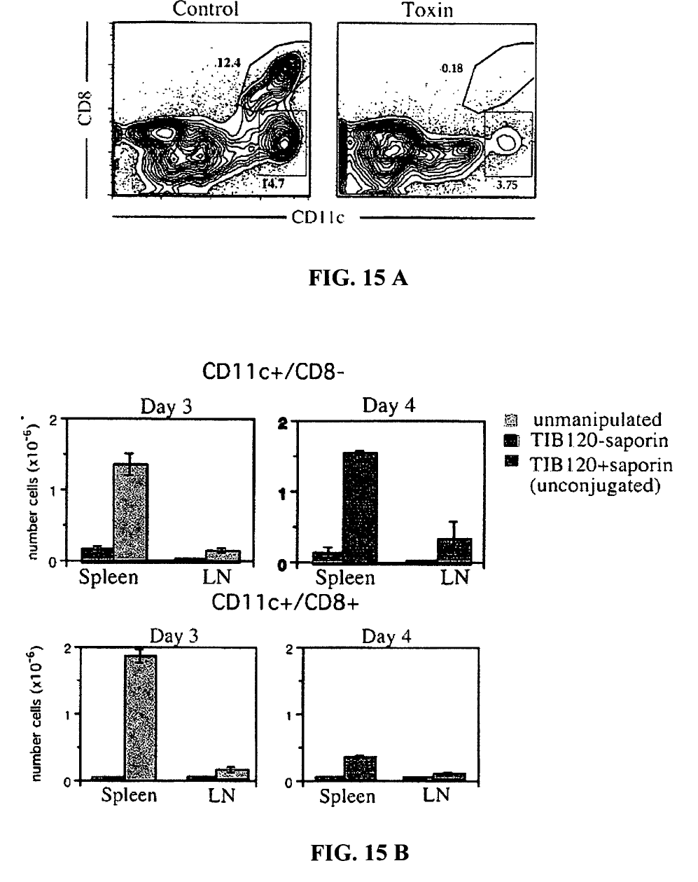

Major Histocompatibility Complex II (MHC II) presents peptide antigens to CD4+ T cells and is expressed on all dendritic cells, as well as B cells and macrophages. An immunotoxin directed against murine MHC II that profoundly depletes murine dendritic cells was developed. Purified anti-MHC II antibody (from hybridoma TIB 120) was conjugated to saporin (the anti-MHC I-saporin immunotoxin conjugate hereinafter referred to as “TB120-saporin”). When TIB120-saporin binds to MHC II, it is internalized and the saporin is hydrolyzed from the immunoglobulin. Once in the cytoplasm, saporin mediates cell death via poisoning ribosomes and blocking protein synthesis. It was first determined that 200 μg of TIB120-saporin can deplete lymph node and splenic dendritic cells when injected intravenously. As shown in FIGS. 15A and 15B, as compared to untreated mice or mice injected with unconjugated TIB120-derived anti-MHC II antibody and saporin, administration of M120-saporin conjugate caused a profound depletion of CD11c⁺CD8⁺ and CD11c⁺CD8⁻ dendritic cells on days 3 and 4 after injection. The result shows that a saporin-conjugated immunotoxin against MHC II depletes dendritic cells using the compositions and assays described above.

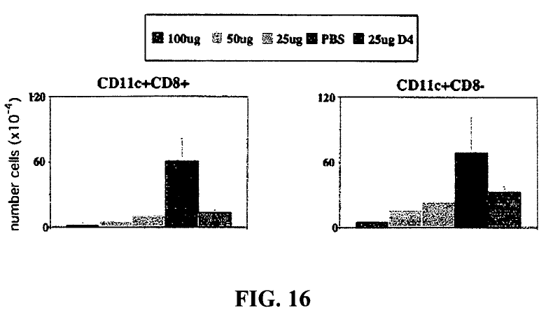

Additionally, a dose titration of a separate preparation of TIB120-saporin was performed. As shown in FIG. 16, a dose-dependent depletion of splenic dendritic cells was observed. The result shows that a saporin-conjugated immunotoxin against MHC II depletes dendritic cells in a dose-dependent manner using the compositions and assays described above.

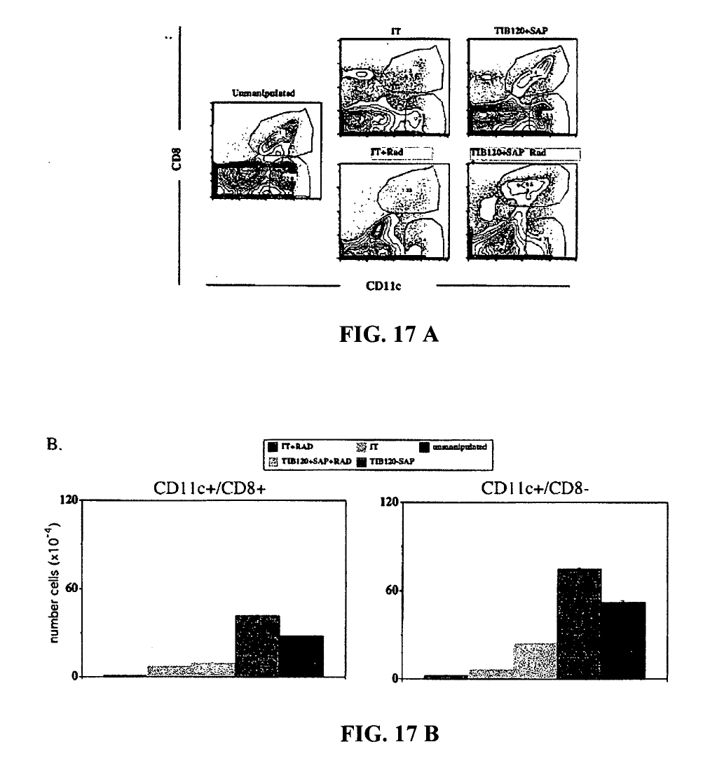

In order to use TIB120-saporin in graft-versus-host disease (GVHD) experiments, TIB120-saporin was co-administered with irradiation to mice. Varying doses of TIB120-saporin were evaluated in conjunction with several irradiation doses to determine which combinations would give sufficient survival for GVHD experiments. It was found that the maximal tolerated combination was 50 μg of TIB120-saporin given 3 days prior to two 450cGy fractions of irradiation. Though 50 μg of immunotoxin was inferior to a 100 μg dose in depleting dendritic cells (FIG. 16) and two 450cGy fractions of irradiation is less than a single dose of 100 cGy typically used in this GVHD model, the addition of 50 μg of immunotoxin to irradiation further depleted dendritic cells as compared with irradiation alone (FIGS. 17A and 17B). The result shows that a saporin-conjugated immunotoxin against MHC II further depletes dendritic cells as compared to irradiation alone using the compositions and assays described above.

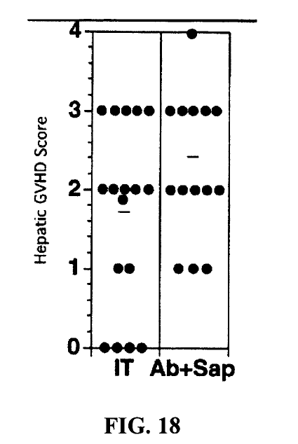

Additionally, the efficacy of a TIB120-saporin conjugate in ameliorating GVHD was examined. Recipient B6 mice received 50 μg of immunotoxin or 50 μg of TIB-120-derived anti-MHC II antibody plus unconjugated saporin (in a 1:1 molar stochiometry) on Day-3. On day 0, mice received two 450cGy fractions of irradiation (separated by 3 hours) and were reconstituted with C3H.SW T cell depleted bone marrow (BM) with or without 2.7×106 C3H.SW CD8⁺ T cells. Overall, only a few mice in each GVHD group were affected by clinical cutaneous GVHD. Mice were sacrificed approximately 40 days post transplant, and tissue was harvested for histologic analysis. Recipients of only T cell depleted bone marrow had no evidence of hepatic GVHD (5 mice/group; not shown). However, recipients of immunotoxin had reduced hepatic GVHD as compared with mice that received TIB-120-derived anti-MHC II antibody plus unconjugated saporin (P<0.079; Mann-Whitney). Although the p value was not less than 0.05, it is still highly likely (a 92% chance) that treatment with immunotoxin reduced hepatic GVHD (FIG. 18). As discussed above, though 50 μg of TIB120-saporin is a suboptimal dose in terms of dendritic cell depletion, a reduction in GVHD is still observed. This makes it highly likely that optimization of the protocol for administering the TIB120-saporin will further decrease GVHD in a host organism (for example, giving multiple doses of toxin pretreating with corticosteroids to reduce toxicity). The result shows that a saporin-conjugated immunotoxin against MHC II decreases pathologic GVHD using the compositions and assays described above.

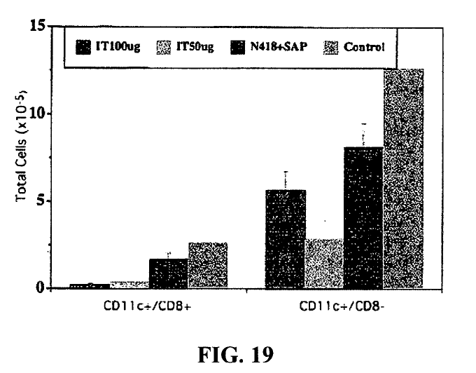

Additionally, a saporin-conjugated immunotoxin directed against marine CD11c was developed. CD11c is a beta-integrin expressed on all subsets of marine dendritic cells. An anti-CD11c antibody was purified from clone N418, which is derived from a fusion of splenocytes from immunized hamsters. Saporin was conjugated to the purified N418-derived anti-CD11c antibody (the anti-CD11c-saporin immunotoxin conjugate hereinafter referred to as “N418-saporin”). The efficacy of N418-saporin was examined by injecting it intravenously and then evaluating dendritic cells in the spleen and lymph nodes as described above, except that because CD11c was blocked by bound N418-saporin or free saporin, instead of staining for CD11c directly, an anti-hamster antibody that recognized the N418-derived anti-CD11c antibody was used. As a control, a cohort of mice was injected with 100 μg of N418-derived anti-CD11c antibody and saporin (unconjugated). As shown in FIG. 19A, N418-saporin resulted in a statistically significant depletion of CD8⁺ and to a lesser extent CD8⁻ dendritic cells as compared with untreated or N418-derived anti-CD11c antibody plus unconjugated saporin treated mice. Results of the statistical analysis of this experiment, presented as Mann-Whitney p values, are set forth in FIG. 19B. The results of the statistical analysis very strongly support the CD11c-targeted depletion of dendritic cells using the N418-saporin immunotoxin conjugate. The result shows that a saporin-conjugated immunotoxin against CD11c depletes dendritic cells in a dose-dependent manner using the compositions and assays described above.

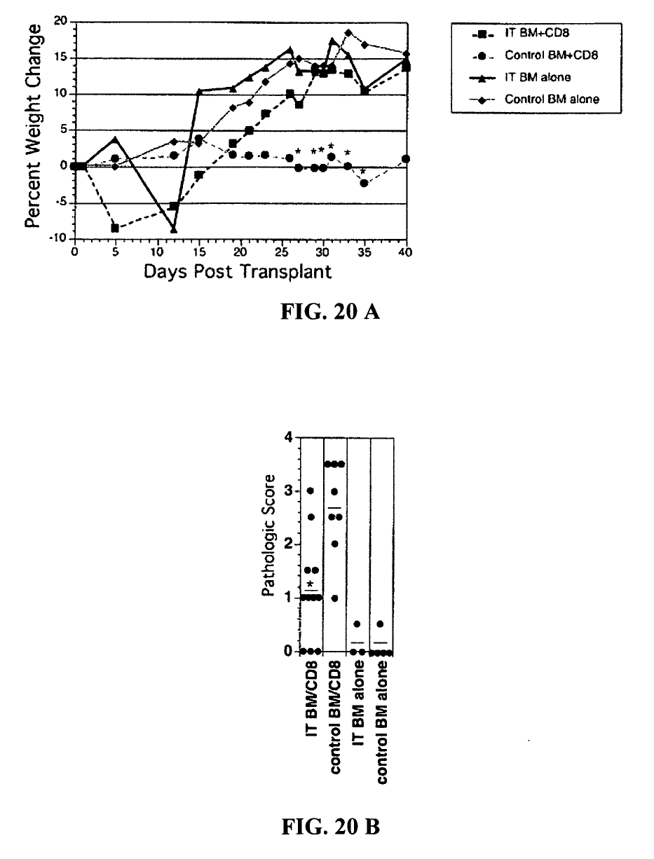

Additionally, the ability of the combined administration of TIB120-saporin and N418-saporin to suppress GVHD was examined. Mice received TIB120-saporin and N418-saporin on day 3. On day 0, they underwent a GVHD-inducing transplant as described above. Strikingly, immunotoxin treated CD8 recipients developed statistically significant less clinical GVHD as shown by percent weight change (FIGS. 20A and 20B). Mice were sacrificed 40 days post transplant and histology was taken. Hepatic GVHD was scored without knowledge as to the experimental group and was verified by two independent observers. As shown in FIG. 20B, hepatic GVHD was significantly reduced in immunotoxin treated CD8 recipients (P<0.006 by Mann-Whitney). Thus, recipient antigen presenting cell (APC) depletion with TIB120-saporin and N418-saporin decreases GVHD both clinically and pathologically.

Further, the role of B cells in GVHD was demonstrated. B cell-deficient B6 muMT or B6 wild type recipients were irradiated and reconstituted with C3H.SW CD8 cells. Mice were sacrificed on day 37 post transplant, and skin, liver, and bowel were harvested, fixed in 10% phosphate buffered formalin, embedded in paraffin, sectioned, and stained with hematoxylin and eosin. Slides were numbered and read by pathologists expert in skin and gastrointestinal disease, and without knowledge as to the experimental group from which each sample was derived. muMT recipients genetically deficient in B cells developed GVHD similar to that observed in wild type recipients. The result shows that host B cells are not required for GVHD induction. Using the compositions and assays disclosed in the above-identified patent application, it has been shown that mice genetically deficient in B-cells and wild type mice have comparable histologic GVHD. It is thus clearly established that a decrease in GVHD is achieved by way of antigen presenting cell depletion in a host according to the compositions and assays disclosed in the above-identified patent application.

In summary, the data presented herein demonstrate that immunotoxins can be used to decrease grain-versus-host disease in a host organism. That is, the data described herein amply support that immunotoxins can be used to deplete antigen-presenting cells in a population of cells in an organism, that host dendritic cells are involved in the graft-versus-host response, and that depletion of dendritic cells in an organism decreases graft-versus-host disease in a host organism. Therefore, the data described herein provide working examples of the suppression of graft-versus-host disease in a mammal, as well as the depletion of antigen presenting cells in a population of hematopoietic cells in a mammal. Further, the data described herein, when taken together, provides guidance as to the degree of antigen presenting cell depletion required to decrease the graft-versus-host response in a host organism.

### REFERENCES

Goker, H., Haznedaroglu, I. C. & Chao, N. J. Acute graft-vs-host disease: Pathobiology and management. Exp. Hematol. 29, 259-277 (2001).|
Shlomchik, W. D. et al. Prevention of graft versus host disease by inactivation of host antigen-presenting cells. Science 285, 412-415 (1999).
Bland, P. W. & Whiting, C.V. Induction of MHC class II gene products in rat intestinal epithelium during graft-versus-host disease and effects on the immune function of the epithelium. Immunology 75, 366-371 (1992).
Matte, C. C. et al. Graft-versus-leukemia in a retrovirally induced murine CML model: mechanisms of T-cell killing. Blood 103, 4353-4361 (2004).
Pear, W. S. et al. Efficient and rapid induction of a chronic myelogenous leukemia-like myeloproliferative disease in mice receiving P210 bcr/abl-transduced bone marrow. Blood 92, 3780-3792 (1998).
Daley, G. Q., Van Etten, R. A. & Baltimore, D. Induction of chronic myelogenous leukemia in mice by the P210bcr/abl gene of the Philadelphia chromosome. Science 247, 824-830 (1990).
Kaech, S. M. & Ahmed, R. Memory CD8+ T cell differentiation: initial antigen encounter triggers a developmental program in naive cells. Nat. Immunol. 2, 415-422 (2001).
van Stipdonk, M. J., Lemmens, E. E. & Schoenberger, S. P. Naive CTLs require a single brief period of antigenic stimulation for clonal expansion and differentiation. Nat. Immunol. 2, 423-429 (2001).
Mintern, J. D. et al. Cutting edge: precursor frequency affects the helper dependence of cytotoxic T cells. J. Immunol. 168, 977-980 (2002).
Iezzi, G., Karjalainen, K. & Lanzavecchia, A. The duration of antigenic stimulation determines the fate of naive and effector T cells. Immunity 8, 89-95 (1998).
Croft, M., Bradley, L. M. & Swain, S. L. Naive versus memory CD4 T cell response to antigen. Memory cells are less dependent on accessory cell costimulation and can respond to many antigen-presenting cell types including resting B cells. J. Immunol. 152, 2675-2685 (1994).

Kedl, R. M. & Mescher, M. F. Qualitative differences between naive and memory T cells make a major contribution to the more rapid and efficient memory CD8+ T cell response. J. Immunol. 161, 674-683 (1998).
Zimmermann, C., Prevost-Blondel, A., Blaser, C. & Pircher, H. Kinetics of the response of naive and memory CD8 T cells to antigen: similarities and differences. Eur. J. Immunol. 29, 284-290 (1999).
Cho, B. K., Wang, C., Sugawa, S., Eisen, H. N. & Chen, J. Functional differences between memory and naive CD8 T cells. Proc. Natl. Acad. Sci. USA 96, 2976-2981 (1999).
London, C. A., Lodge, M. P. & Abbas, A. K. Functional responses and costimulator dependence of memory CD4+ T cells. J. Immunol. 164, 265-72 (2000).
Sprent, J. T memory cells: quality not quantity. Curr. Biol. 12, R174-R176 (2002).
Le Bon, A. et al. Cross-priming of CD8(+) T cells stimulated by virus-induced type I interferon. Nat. Immunol. 4, 1009-1015 (2003).
Singh-Jasuja, H. et al. The heat shock protein gp96 induces maturation of dendritic cells and down-regulation of its receptor. Eur. J. Immunol. 30, 2211-2215 (2000).
Hu, H. M. et al. CD28, TNF receptor, and IL-12 are critical for CD4-independent cross-priming of therapeutic antitumor CD8+ T cells. J. Immunol. 169, 4897-4904 (2002).
Hoffmann, T. K., Meidenbauer, N., Muller-Berghaus, J., Storkus, W. J. & Whiteside, T. L. Proinflammatory cytokines and CD40 ligand enhance cross-presentation and cross-priming capability of human dendritic cells internalizing apoptotic cancer cells. J. Immunother. 24, 162-171 (2001).
Kopp, E. & Medzhitov, R. Recognition of microbial infection by Toll-like receptors. Curr. Opin. Immunol. 15, 396-401 (2003).
Horowitz, M. M. et al. Graft-versus-leukemia reactions after bone marrow transplantation. Blood 75, 555-562 (1990).
Kolb, H. J. et al. Graft-versus-leukemia effect of donor lymphocyte transfusions in marrow grafted patients. European Group for Blood and Marrow Transplantation Working Party Chronic Leukemia. Blood 86, 2041-2050 (1995).
Choudhury, A. et al. Use of leukemic dendritic cells for the generation of antileukemic cellular cytotoxicity against Philadelphia chromosome-positive chronic myelogenous leukemia. Blood 89, 1133-1142 (1997).
Chakrabarti, S. et al. High incidence of cytomegalovirus infection after nonmyeloablative stem cell transplantation: potential role of Campath-1H in delaying immune reconstitution. Blood 99, 4357-4363 (2002).
Cragg, L. et al. A randomized trial comparing prednisone with antithymocyte globulin/prednisone as an initial systemic therapy for moderately severe acute graft-versus-host disease. Biol. Blood Marrow Transplant. 6, 441-447 (2000).
Bix, M. et al. Rejection of class I MHC-deficient haemopoietic cells by irradiated MHC-matched mice. Nature 349, 329-331 (1991).|
Anderson, B. E. et al. Memory CD4+ T cells do not induce graft-versus-host disease. J. Clin. Invest. 112, 101-108 (2003).|
Pear, W. S., Nolan, G. P., Scott, M. L. & Baltimore, D. Production of high-titer helper-free retroviruses by transient transfection. Proc. Natl. Acad. Sci. USA 90, 8392-8396 (1993).
Lee S J, Klein J P, Barrett A J, et al. Severity of chronic graft-versus-host disease: association with treatment-related mortality and relapse. Blood. 2002; 100: 406-414.
Anderson D, DeFor T, Burns L, et al. A comparison of related donor peripheral blood and bone marrow transplants: importance of late-onset chronic graft-versus-host disease and infections. Biol Blood Marrow Transplant. 2003; 9: 52-59.
Atkinson K, Horowitz M M, Gale R P, et al. Risk factors for chronic graft-versus-host disease after HLA-identical sibling bone marrow transplantation. Blood. 1990; 75: 2459-2464.
Castagna L, Bertuzzi A, Nozza A, et al. Reduced intensity conditioning regimen followed by glycosylated G-CSF mobilized PBSCT in patients with solid tumors and malignant lymphomas. Bone Marrow Transplant. 2002; 30: 207-214.
Flowers M E, Parker P M, Johnston L J, et al. Comparison of chronic graft-versus-host disease after transplantation of peripheral blood stem cells versus bone marrow in allogeneic recipients: long-term follow-up of a randomized trial. Blood. 2002; 100: 415-419.
Remberger M, Kumlien G, Aschan J, et al. Risk factors for moderate-to-severe chronic graft-versus-host disease after allogeneic hematopoietic stem cell transplantation. Biol Blood Marrow Transplant. 2002; 8: 674-682.
Zecca M, Prete A, Rondelli R, et al. Chronic graft-versus-host disease in children: incidence, risk factors, and impact on outcome. Blood. 2002; 100: 1192-1200.
Auffermann-Gretzinger S, Lossos I S, Vayntrub T A, et al. Rapid establishment of dendritic cell chimerism in allogeneic hematopoietic cell transplant recipients. Blood. 2002; 99: 1442-1448
Clark F J, Freeman L, Dzionek A, et al. Origin and subset distribution of peripheral blood dendritic cells in patients with chronic graft-versus-host disease. Transplantation. 2003; 75: 221-225.
Imamura M, Tsutsumi Y, Miura Y, Toubai T, Tanaka J. Immune reconstitution and tolerance after allogeneic hematopoietic stem cell transplantation. Hematology. 2003; 8: 19-26.
Nachbaur D, Kircher B, Eisendle K, Latzer K, Haun M, Gastl G. Phenotype, function and chimaerism of monocyte-derived blood dendritic cells after allogeneic haematopoietic stem cell transplantation. Br J Haematol. 2003; 123: 119-126.
Shlomchik W D, Couzens M S, Tang C B, et al. Prevention of graft versus host disease by inactivation of host antigen-presenting cells. Science. 1999; 285: 412-415.
Ruggeri L, Capanni M, Urbani E, et al. Effectiveness of donor natural killer cell alloreactivity in mismatched hematopoietic transplants. Science. 2002; 295: 2097-2100.
Hamilton B L. L3T4-positive T cells participate in the induction of graft-vs-host disease in response to minor histocompatibility antigens. J Immunol. 1987; 139: 2511-2515.
Germain R N. MHC-dependent antigen processing and peptide presentation: providing ligands for T lymphocyte activation. Cell. 1994; 76: 287-299. [
Lanzavecchia A. Mechanisms of antigen uptake for presentation. Curr Opin Immunol. 1996; 8: 348-354.
Sant A J. Endogenous antigen presentation by MHC class II molecules. Immunol Res. 1994; 13: 253-267.
Wolf P R, Ploegh H L. How MHC class II molecules acquire peptide cargo: biosynthesis and trafficking through the endocytic pathway. Annu Rev Cell Dev Biol. 1995; 11: 267-306.
Brooks A G, McCluskey J. Class II-restricted presentation of a hen egg lysozyme determinant derived from endogenous antigen sequestered in the cytoplasm or endoplasmic reticulum of the antigen presenting cells. J. Immunol. 1993; 150: 3690-3697.
Bogen B, Weiss S. Processing and presentation of idiotypes to MHC-restricted T cells. Int Rev Immunol. 1993; 10: 337-355.
Bodmer H, Viville S, Benoist C, Mathis D. Diversity of endogenous epitopes bound to MHC class II molecules limited by invariant chain. Science. 1994; 263: 1284-1286.
Lechler R, Aichinger G, Lightstone L. The endogenous pathway of MHC class II antigen presentation. Immunol Rev. 1996; 151: 51-79.
Li J, Helm K, Howell C D. Contributions of donor CD4 and CD8 cells to liver injury during murine graft-versus-host disease. Transplantation. 1996; 62: 1621-1628.
Nonomura A, Kono N, Mizukami Y, Nakanuma Y. Histological changes of the liver in experimental graft-versus-host disease across minor histocompatibility barriers, VII: a light and electron microscopic study of the large bile duct. Liver. 1993; 13: 163-171.
Vierling J M, Ruderman W B, Jaffee B D, Fennell R H Jr, Claman H N. Hepatic lesions in murine chronic graft-versus-host disease to minor histocompatibility antigens: a reproducible model of nonsuppurative destructive cholangitis. Transplantation. 1989; 48: 717-718.
McCormick L L, Zhang Y, Tootell E, Gilliam A C. Anti-TGF-beta treatment prevents skin and lung fibrosis in murine sclerodermatous graft-versus-host disease: a model for human scleroderma. J. Immunol. 1999; 163: 5693-5699.
Borriello F, Sethna M P, Boyd S D, et al. B7-1 and B7-2 have overlapping, critical roles in immunoglobulin class switching and germinal center formation. Immunity. 1997; 6: 303-313.
Anderson B E, McNiff J, Matte C, Athanasiadis I, Shlomchik W D, Shlomchik M J. Recipient CD4+ T cells that survive irradiation regulate chronic graft-versus-host disease. Blood. 2004; 104: 1565-1573.
Anderson B E, McNiff J, Yan J, et al. Memory CD4+ T cells do not induce graft-versus-host disease. J Clin Invest. 2003; 112: 101-108.
Kenty G, Bikoff E K. BALB/c invariant chain mutant mice display relatively efficient maturation of CD4+ T cells in the periphery and secondary proliferative responses elicited upon peptide challenge. J. Immunol. 1999; 163: 232-241.
Merad M, Hoffmann P, Ranheim E, et al. Depletion of host Langerhans cells before transplantation of donor alloreactive T cells prevents skin graft-versus-host disease. Nat Med. 2004; 10: 510-517.
Salomon B, Lenschow D J, Rhee L, et al. B7/CD28 costimulation is essential for the homeostasis of the CD4+ CD25+immunoregulatory T cells that control autoimmune diabetes. Immunity. 2000; 12: 431-440.
Salomon B, Bluestone J A. Complexities of CD28/B7: CTLA-4 costimulatory pathways in autoimmunity and transplantation. Annu Rev Immunol. 2001; 19: 225-252.
Blazar B R, Taylor P A, Panoskaltsis-Mortari A, et al. Blockade of CD40 ligand-CD40 interaction impairs CD4+ T cell-mediated alloreactivity by inhibiting mature donor T cell expansion and function after bone marrow transplantation. J Immunol. 1997; 158: 29-39.
Korngold R, Sprent J. Lethal GVHD across minor histocompatibility barriers: nature of the effector cells and role of the H-2 complex. Immunol Rev. 1983; 71: 5-29.
Teshima T, Ordemann R, Reddy P, et al. Acute graft-versus-host disease does not require alloantigen expression on host epithelium. Nat Med. 2002; 8: 575-581.
Duffner U A, Maeda Y, Cooke K R, et al. Host dendritic cells alone are sufficient to initiate acute graft-versus-host disease. J. Immunol. 2004; 172: 7393-7398.
Carbone F R, Bevan M J. Class I-restricted processing and presentation of exogenous cell-associated antigen in vivo. J Exp Med. 1990; 171: 377-387.
Jones S C, Murphy G F, Friedman T M, Korngold R. Importance of minor histocompatibility antigen expression by nonhematopoietic tissues in a CD4+ T cell-mediated graft-versus-host disease model. J Clin Invest. 2003; 112: 1880-1886.
Green J M, Noel P J, Sperling A I, et al. Absence of B7-dependent responses in CD28-deficient mice. Immunity. 1994; 1: 501-508.
Sperling A I, Auger J A, Ehst B D, Rulifson I C, Thompson C B, Bluestone J A. CD28/B7 interactions deliver a unique signal to naive T cells that regulates cell survival but not early proliferation. J Immunol. 1996; 157: 3909-3917.
Schweitzer A N, Sharpe A H. Studies using antigen-presenting cells lacking expression of both B7-1 (CD80) and B7-2 (CD86) show distinct requirements for B7 molecules during priming versus restimulation of Th2 but not Th1 cytokine production. J Immunol. 1998; 161: 2762-2771.
Chen B J, Cui X, Sempowski G D, Liu C, Chao N J. Transfer of allogeneic CD62L-memory T cells without graft-versus-host disease. Blood. 2004; 103: 1534-1541.
Lumsden J M, Prasad S J, Peach R J, Ronchese F. The effects of B7-dependent costimulation on T cell division and survival in vivo and in vitro are dependent on antigen concentration. Eur J Immunol. 2003; 33: 2074-2082.
Speiser D E, Bachmann M F, Shahinian A, Mak T W, Ohashi P S. Acute graft-versus-host disease without costimulation via CD28. Transplantation. 1997; 63: 1042-1044.
Yu X Z, Martin P J, Anasetti C. Role of CD28 in acute graft-versus-host disease. Blood. 1998; 92: 2963-2970.
Via C S, Rus V, Nguyen P, Linsley P, Gause W C. Differential effect of CTLA4Ig on murine graft-versus-host disease (GVHD) development: CTLA4Ig prevents both acute and chronic GVHD development but reverses only chronic GVHD. J Immunol. 1996; 157: 4258-4267.
Blazar B R, Sharpe A H, Taylor P A, et al. Infusion of anti-B7.1 (CD80) and anti-B7.2 (CD86) monoclonal antibodies inhibits murine graft-versus-host disease lethality in part via direct effects on CD4+ and CD8+ T cells. J Immunol. 1996; 157: 3250-3259.
Blazar B R, Taylor P A, Linsley P S, Vallera D A. In vivo blockade of CD28/CTLA4: B7/BB1 interaction with CTLA4-Ig reduces lethal murine graft-versus-host disease across the major histocompatibility complex barrier in mice. Blood. 1994; 83: 3815-3825.
Blazar B R, Taylor P A, Gray G S, Vallera D A. The role of T cell subsets in regulating the in vivo efficacy of CTLA4-Ig in preventing graft-versus-host disease in recipients of fully MHC or multiple minor histocompatibility-disparate donor inocula. Transplantation. 1994; 58: 1422-1426.
Inaba K, Inaba M, Witmer-Pack M, Hatchcock K, Hodes R, Steinman R M. Expression of B7 costimulator molecules on mouse dendritic cells. Adv Exp Med Biol. 1995; 378: 65-70.
Caux C, Massacrier C, Vanbervliet B, et al. Activation of human dendritic cells through CD40 cross-linking. J Exp Med. 1994; 180: 1263-1272.
Ridge J P, Di Rosa F, Matzinger P. A conditioned dendritic cell can be a temporal bridge between a CD4+ T-helper and a T-killer cell. Nature. 1998; 393: 474-478
Antin J H, Ferrara J L. Cytokine dysregulation and acute graft-versus-host disease. Blood. 1992; 80: 2964-2968
Ferrara J L, Abhyankar S, Gilliland D G. Cytokine storm of graft-versus-host disease: a critical effector role for interleukin-1. Transplant Proc. 1993; 25: 1216-1217.
Matte C C, Liu J, Cormier J, et al. Donor APCs are required for maximal GVHD but not for GVL. Nat Med. 2004; 10: 987-992.

The disclosures of each and every publication (e.g., books, scientific articles, treatises, letters to the editor, theses, notes, reviews, etc.), patent and patent application discussed herein are incorporated by reference.

While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method of treating graft versus host disease (GVHD) in a host mammal, said method comprising:
(a) transferring hematopoietic cells from a donor mammal to said host mammal; and
(b) depleting and/or inactivating antigen presenting cells in a population of hematopoietic cells in said host mammal with an antigen presenting cell depleting and/or inactivating composition, wherein said graft versus host disease is treated in said host mammal by virtue of said depletion and/or inactivation of said antigen presenting cells.

2. The method of claim 1, wherein said antigen presenting cells are donor cells.

3. The method of claim 1, wherein said antigen presenting cells are host cells.

4. The method of claim 1, wherein said antigen presenting cells are selected from the group consisting of dendritic cells, B lymphocytes, macrophages, monocytes, CD34⁺ cells, fibroblasts, stem cells, and cheratinocytes.

5. The method of claim 1, wherein said host is human.

6. The method of claim 1, wherein said antigen presenting cell depleting and/or inactivating composition is selected from the group consisting of a toxin, an antibody, a radioactive molecule, a nucleic acid, a peptide, a peptidomemetic and a ribozyme.

7. The method of claim 6, wherein said toxin is an immunotoxin.

8. The method of claim 6, wherein said toxin is selected from the group consisting of ricin, diptheria toxin and pseudomonas exotoxin A and saporin.

9. The method of claim 8, wherein the toxin is saporin.

10. The method of claim 6, wherein said antibody is selected from the group consisting of antibody specific for CD1a, antibody specific for CD11c, antibody specific for MHCII, antibody specific for CD11b, antibody specific for DEC205, antibody specific for B71, antibody specific for B72, antibody specific for CD40, antibody specific for Type I lectins and antibody specific for Type II lectins.

11. The method of claim 6, wherein said nucleic acid molecule is selected from the group consisting of a gene and an oligonucleotide.

12. The method of claim 6, wherein said radioactive molecule is a radioactively labeled antibody.

13. The method of claim 6, wherein said antigen presenting cell depleting and/or inactivating composition is a chimeric composition comprising an antibody and a toxin.

14. The method of claim 13, wherein said toxin is selected from the group consisting of ricin, diptheria toxin and pseudomonas exotoxin A and saporin.

15. The method of claim 14, wherein said toxin is saporin.

16. The method of claim 13, wherein said antibody is selected from the group consisting of antibody specific for CD1a, antibody specific for CD11c, antibody specific for MHCII, antibody specific for CD11b, antibody specific for DEC205, antibody specific for B71, antibody specific for B72, antibody specific for CD40, antibody specific for Type I lectins and antibody specific for Type II lectins.

17. The method of 1, wherein said antigen depleting and/or inactivating composition is delivered to said antigen presenting cell in a vector selected from the group consisting of a viral vector and a non-viral vector.

18. A method of preventing and/or decreasing graft versus host disease (GVHD) in a host mammal, said method comprising:
(a) transferring hematopoietic cells from a donor mammal to said host mammal; and
(b) depleting and/or inactivating antigen presenting cells in a population of hematopoietic cells in said host mammal with an antigen presenting cell depleting and/or inactivating composition, wherein said graft versus host disease is prevented and/or decreased in said host mammal by virtue of said depletion and/or inactivation of antigen presenting cells.

19. The method of claim 18, wherein said antigen presenting cells are donor cells.

20. The method of claim 18, wherein said antigen presenting cells are host cells.

21. The method of claim 18, wherein said antigen presenting cells are selected from the group consisting of dendritic cells, B lymphocytes, macrophages, monocytes, CD34⁺ cells, fibroblasts, stem cells, and cheratinocytes.

22. The method of claim 18, wherein said host is human.

23. The method of claim 18, wherein said antigen presenting cell depleting and/or inactivating composition is selected from the group consisting of a toxin, an antibody, a radioactive molecule, a nucleic acid, a peptide, a peptidomemetic and a ribozyme.

24. The method of claim 23, wherein said toxin is an immunotoxin.

25. The method of claim 23, wherein said toxin is selected from the group consisting of ricin, diptheria toxin and pseudomonas exotoxin A and saporin.

26. The method of claim 25, wherein the toxin is saporin.

27. The method of claim 23, wherein said antibody is selected from the group consisting of antibody specific for CD1a, antibody specific for CD11c, antibody specific for MHCII, antibody specific for CD11b, antibody specific for DEC205, antibody specific for B71, antibody specific for B72, antibody specific for CD40, antibody specific for Type I lectins and antibody specific for Type II lectins.

28. The method of claim 23, wherein said nucleic acid molecule is selected from the group consisting of a gene and an oligonucleotide.

29. The method of claim 23, wherein said radioactive molecule is a radioactively labeled antibody.

30. The method of claim 23 wherein said antigen presenting cell depleting composition is a chimeric composition comprising an antibody and a toxin.

31. The method of claim 30, wherein said toxin is selected from the group consisting of ricin, diptheria toxin and pseudomonas exotoxin A and saporin.

32. The method of claim 31, wherein said toxin is saporin.

33. The method of claim 30, wherein said antibody is selected from the group consisting of antibody specific for CD1a, antibody specific for CD11c, antibody specific for MHCII, antibody specific for CD11b, antibody specific for DEC205, antibody specific for B71, antibody specific for B72, antibody specific for CD40, antibody specific for Type I lectins and antibody specific for Type II lectins.

34. The method of 23, wherein said antigen depleting and/or inactivating composition is delivered to said antigen presenting cell in a vector selected from the group consisting of a viral vector and a non-viral vector.
